(19) United States
(12) Patent Application Publication
(10) Pub. No.: US 2012/0065165 A1
(43) Pub. Date: Mar. 15, 2012

(54) COMPOSITIONS AND METHODS OF TREATING ENDOTHELIAL DISORDERS

(75) Inventors: Simon E. Aspland, Baltimore, MD (US); Dan E. Berkowitz, Baltimore, MD (US); Trinity Bivalacqua, Baltimore, MD (US); Hunter Champion, Baltimore, MD (US)

(73) Assignee: ARGINETIX, INC, Lutherville, MD (US)

(21) Appl. No.: 13/127,091

(22) PCT Filed: Nov. 2, 2009
(86) PCT No.: PCT/US09/05923

§ 371 Date: Nov. 28, 2011

Related U.S. Application Data
(60) Provisional application No. 61/110,025, filed on Oct. 31, 2008.

(51) Int. Cl. A61K 31/69; A61K 31/50; A61K 31/4985; A61K 31/501; A61K 31/496; A61K 31/5025; A61K 31/519; A61K 31/522; A61K 31/506; A61K 31/4375; A61K 31/436; A61K 31/437; A61P 11/06; A61P 9/00; A61P 15/10; A61P 29/00; A61P 9/12; A61K 31/53

(52) U.S. Cl. 514/64; 514/246; 514/247; 514/250; 514/252.03; 514/252.04; 514/252.16; 514/253.07; 514/257; 514/262.1; 514/263.32; 514/275; 514/283; 514/291; 514/293; 514/300

(57) ABSTRACT

Compositions and methods of treating endothelial disorders, including: asthma, erectile dysfunction, pulmonary hypertension, cardiovascular disorders, and other disorders, using a synergistic combination of at least one arginase inhibitor and at least one phosphodiesterase (PDE1), (PDE2) and/or (PDE5) inhibitor are described.

### CROSS REFERENCE TO RELATED APPLICATIONS

This application claims benefit of U.S. Provisional Application No. 61/110,025, filed Oct. 31, 2008, which is hereby incorporated by reference in its entirety and relied upon.

### FIELD

The application relates to the combination of a synergistically-effective amount of at least one arginase inhibitor and at least one phosphodiesterase PDE1, PDE2 and/or PDE5 inhibitor and the use of such a combination for the treatment of endothelial disorders, including asthma, cardiovascular disorders, erectile dysfunction, female sexual dysfunction, inflammation, intermittent claudication, peripheral arterial occlusive disorders, pulmonary hypertension, Raynaud's disease, stroke and systemic hypertension. A subset of the patient population having these conditions responds poorly, if at all, to the administration of individual arginase inhibitors or PDE inhibitors. The response of these patients to a synergistic combination of at least one arginase inhibitor and at least one PDE inhibitor is more than additive relative to their response to either inhibitor alone. Arginase and PDE1, PDE2 and/or PDE5 can be synergistically inhibited because these enzymes control endothelial function through a common signaling pathway and in the pathological conditions cited herein, arginase is activated, or up-regulated, at a localized site-specific level. It is at these sites that a synergistic effect from the administration of an arginase inhibitor and a PDE inhibitor is observed.

### BACKGROUND

The independent use of arginase inhibitors and phosphodiesterase has been described for a variety of conditions.

### SUMMARY

This application relates to the combined use of arginase inhibitors with PDE1, PDE2 and/or PDE5 inhibitors, which act synergistically in the wide range of endothelial conditions in which arginase activity is pathologically elevated.

It was recognized that site specificity and spatial confinement are important. Arginase inhibition and PDE inhibition both need to occur in the same organ, or the same spatially-confined area. In the normal population, arginase does not limit the availability of L-arginine as a substrate for nitric oxide synthase to such an extent as to become a limiting factor in nitric oxide (NO) production and the use of an arginase inhibitor has little or no effect on NO production. However, in the pathological conditions cited herein, arginase is activated, or up-regulated, at a localized site-specific level. It is at these sites that a synergistic effect from the administration of an arginase inhibitor and a PDE inhibitor is observed.

In an embodiment, compositions comprise at least one arginase inhibitor and at least one PDE inhibitor. In another embodiment, such compositions are used in methods for treating endothelial disorders, including asthma, cardiovascular disorders, erectile dysfunction, female sexual dysfunction, inflammation, intermittent claudication, peripheral arterial occlusive disorders, pulmonary hypertension, Raynaud's disease, stroke, systemic hypertension, combinations thereof and the like.

In another embodiment, a composition comprises a therapeutically-effective amount of a synergistically-effective combination of at least one arginase inhibitor and at least one phosphodiesterase (PDE) inhibitor formulated in a physiologically-acceptable pharmaceutical medium.

In a further embodiment, the at least one arginase inhibitor in the composition is 2(S)-Amino-6-boronohexanoic acid (ABH), S-(2-boronoethyl)-L-cysteine (BEC), Nω-hydroxy-nor-L-arginine (nor-NOHA), Nω-hydroxy-L-arginine (NOHA), combinations thereof and the like.

In yet another embodiment, the at least one PDE inhibitor in the composition is a PDE1 inhibitor, a PDE2 inhibitor, a PDE5 inhibitor, a non-specific PDE inhibitor that inhibits PDE1, PDE2 and/or PDE5, combinations thereof and the like.

In a further embodiment, the PDE 1 inhibitor in the composition is 5E3623, BAY 383045, HFV 1017, KF 19514, SCH 51866, combinations thereof and the like.

In another embodiment, the PDE2 inhibitor in the composition is BAY 607550.

In yet another embodiment, the PDE5 inhibitor in the composition is mirodenafil, sildenafil, tadalafil, udenafil, vardenafil, avanafil, dasantafil, NM 702, SLX 101, UK 369003, combinations thereof and the like.

In a further embodiment, the non-specific PDE inhibitor in the composition that inhibits PDE1, PDE2 and/or PDE5 is amlexanox, caffeine citrate, doxofylline, levosimendan, mopidamol, pentoxifylline, pemobendan, propentofylline, vesnarinone, ibudilast, combinations thereof and the like.

In yet another embodiment, a kit comprises a formulation comprising a unit dose of at least one arginase inhibitor, and at least one PDE inhibitor, combinations thereof and the like, and a pharmaceutically acceptable excipient to administer the dosage form according to a desired regimen or exemplary regimen, said kit optionally comprising instructions for the use of the kit.

In an embodiment, a method of treating an endothelial disorder is provided, where the method comprises administering to a patient in need thereof a synergistically-effective amount of at least one arginase inhibitor and at least one phosphodiesterase (PDE) inhibitor.

In another embodiment, the endothelial disorder treated is asthma, a cardiovascular disorder, erectile dysfunction, female sexual dysfunction, inflammation, intermittent claudication, a peripheral arterial occlusive disorder, pulmonary hypertension, Raynaud's disease, stroke, systemic hypertension, combinations thereof and the like.

In yet another embodiment, the at least one arginase inhibitor used in treating an endothelial disorder is 2(S)-Amino-6-boronohexanoic acid (ABH), S-(2-boronoethyl)-L-cysteine (BEC), Nω-hydroxy-nor-L-arginine (nor-NOHA), Nω-hydroxy-L-arginine (NOHA), combinations thereof and the like.

In still another embodiment, the at least one PDE inhibitor used in treating an endothelial disorder is a PDE1 inhibitor, a PDE2 inhibitor, a PDE5 inhibitor, a non-specific PDE inhibitor that inhibit PDE1, PDE2 and/or PDE5, combinations thereof and the like.

In another embodiment, the PDE 1 inhibitor used in treating an endothelial disorder is 5E3623, BAY 383045, HFV 1017, KF 19514, SCH 51866, or a combination thereof.

In a further embodiment, the PDE2 inhibitor used in treating an endothelial disorder is BAY 607550.

In another embodiment, the PDE5 inhibitors used in treating an endothelial disorder is mirodenafil, sildenafil, tadalafil, udenafil, vardenafil, avanafil, dasantafil, NM 702, SLX 101, UK 369003, combinations thereof and the like.

In yet another embodiment, the non-specific PDE inhibitor used in treating an endothelial disorder that inhibits PDE1, PDE2 and/or PDE5 is amlexanox, caffeine citrate, doxofylline, levosimendan, mopidamol, pentoxifylline, pemobendan, propentofylline, vesnarinone, ibudilast, combinations thereof and the like.

In an embodiment, a synergistically-effective amount of at least one arginase inhibitor and at least one phosphodiesterase (PDE) inhibitor is administered together in a single composition in treating an endothelial disorder.

In another embodiment, the synergistically-effective amount of at least one arginase inhibitor and at least one phosphodiesterase (PDE) inhibitor is administered in separate compositions in treating an endothelial disorder.

In yet another embodiment, the synergistically-effective amount of at least one arginase inhibitor and at least one phosphodiesterase (PDE) inhibitor is administered by at least one route of oral, inhalation, intranasal and topical in treating asthma.

In still a further embodiment, the synergistically-effective amount of at least one arginase inhibitor and at least one phosphodiesterase (PDE) inhibitor is administered via oral, topical or injection in treating erectile dysfunction or female sexual dysfunction.

In an embodiment, the synergistically-effective amount of at least one arginase inhibitor and at least one phosphodiesterase (PDE) inhibitor is administered orally in treating a cardiovascular disorder.

In a further embodiment, a regime or regime for treating an endothelial disorder is provided, where the regime or regime comprises administering to a patient in need thereof a synergistically-effective amount of at least one arginase inhibitor and at least one phosphodiesterase (PDE) inhibitor for a specified time at a specified dosing schedule.

In another embodiment, a synergistically-effective amount of at least one arginase inhibitor and at least one phosphodiesterase (PDE) inhibitor is used in the preparation of a medicament for the treatment of an endothelial disorder.

In yet another embodiment, a synergistically-effective amount of at least one arginase inhibitor and at least one phosphodiesterase (PDE) inhibitor is used in the preparation of a medicament for the treatment of an endothelial disorder where the endothelial disorder is asthma, a cardiovascular disorder, erectile dysfunction, female sexual dysfunction, inflammation, intermittent claudication, a peripheral arterial occlusive disorder, pulmonary hypertension, Raynaud's disease, stroke, systemic hypertension or a combination thereof.

### BRIEF DESCRIPTION OF THE DRAWINGS

FIG. 1: Schematics of PDE regulation of NOS-NO generated cGMP in a vascular smooth muscle cell and cardiac myocyte.

FIG. 2: Competitive utilization of L-arginine as a substrate by either arginase or eNOS.

FIG. 3: Schematic of ABH.

FIG. 4: Increase in ICP/MAP (A) and total ICP (B; area under the erectile curve) in response to cavernous nerve stimulation (CNS) in aged rats and aged rats treated with ABH (6 mg/kg) in the drinking water for 28 days.

FIG. 5: Reduction in vascular stiffness and reversal of endothelial dysfunction in old Fisher rats due to arginase inhibition with ABH.

FIG. 6: Enhanced NO production and decreased ROS in aorta of old rats due to arginase inhibition.

FIG. 7: Arginase II (Arg 2) protein expression in human corpus cavernosum from control and diabetic men by Western blot analysis.

FIG. 8: Penile arginase activity in rat penes 2 months after the induction of type 1 diabetes vs. age-matched controls.

FIG. 9: Schematic representation of synergistic interaction between ABH and PDE5 inhibitors

### DETAILED DESCRIPTION

### Definitions

“Arginine” or “Arg” or “L-Arg” as used herein refers to naturally-occurring or synthetically-produced L-arginine, combinations thereof and the like.

“Arginase” as used herein refers to an enzyme that mediates conversion of L-Arg into ornithine and urea, and is meant to encompass any or all relevant arginase types, including, for example, arginase type I, arginase type II, combinations thereof and the like.

“Arginase inhibitor” refers to an agent, such an organic compound or anti-arginase antibody, which agent can be either naturally-occurring or synthetic, which agent affects activity of an arginase (e.g., arginase type I, arginase type II, or both) in catalysis of L-Arg into ornithine and urea. For example, an antibody which binds arginase can affect arginase activity by interfering with arginase binding to its substrate or by promoting clearance of arginase from the subject's circulation.

ABH refers to the arginase inhibitor: 2(S)-Amino-6-boronohexanoic acid.

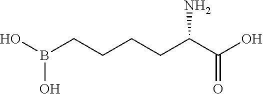

BEC refers to the arginase inhibitor: S-(2-Boronoethyl)-L-cysteine.

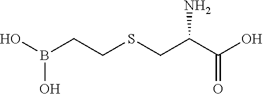

“Phosphodiesterase inhibitor” or “PDE inhibitor” refers to any compound that inhibits the enzyme phosphodiesterase. The term refers to selective or non-selective inhibitors of cyclic guanosine 3′,5′-monophosphate phosphodiesterases (cGMP-PDE), cyclic adenosine 3′,5′-monophosphate phosphodiesterases (cAMP-PDE), combinations thereof and the like.

“Synergistic” refers to an affect that results from two or more agents working together to produce a result not obtainable by any of the agents independently. That result is more than the sum of the results observed when each agent is used independently. Such synergy is advantageous in that it allows for each therapeutic agent typically to be administered in an amount less than if the combined therapeutic effects were additive. Thus, therapy can be effected for patients who, for example, do not respond adequately to the use of one component at what would be considered a maximum strength dose. Additionally, by administering the components in lower amounts relative to the case where the combined effects are additive, side effects such as any priapism or pain at the site of injection can be minimized or avoided in many cases. Such synergy can be demonstrated by the tests disclosed below.

“Therapeutically effective amount” refers to the amount of the at least one arginase inhibitor and the at least one PDE inhibitor that is effective to achieve its intended purpose. While individual patient needs can vary, determination of optimal ranges for effective amounts of each of the compounds and compositions is within the skill of the art. Generally, the dosage required to provide an effective amount of the composition, and which can be adjusted by one of ordinary skill in the art will vary, depending on the age, health, physical condition, sex, weight, extent of the dysfunction of the recipient, frequency of treatment and the nature and scope of the dysfunction.

“Synergistically-effective amount” refers to the amount of the at least one arginase inhibitor and the at least one PDE inhibitor that is effective to achieve its intended purpose. While individual patient needs can vary, determination of optimal ranges for effective amounts of each of the compounds and compositions is within the skill of the art. Generally, the dosage required to provide a synergistically-effective amount of the composition, and which can be adjusted by one of ordinary skill in the art, will vary depending on the age, health, physical condition, sex, weight, extent of the dysfunction of the recipient, frequency of treatment, the nature and scope of the dysfunction and the method by which the inhibitors are administered.

The exact dose of each component administered will, of course, differ depending on the specific components prescribed, on the subject being treated, on the severity of the disease or condition, on the manner of administration and on the judgment of the prescribing physician. Thus, because of patient-to-patient variability, the dosages given below are a guideline and the physician may adjust doses of the compounds to achieve the treatment that the physician considers appropriate for the patient, male or female. In considering the degree of treatment desired, the physician must balance a variety of factors such as the age of the patient and the presence of other diseases or conditions (e.g., cardiovascular disease). The usual doses of the arginase inhibitors and the PDE inhibitors are each about 0.001 mg to about 1500 mg per day, preferably about 1 mg to about 1000 mg per day, more preferably about 10 mg to about 750 mg per day. Table 1 shows the doses of PDE5 inhibitors that have been utilized in man to treat either erectile dysfunction or pulmonary arterial hypertension. Thus, for ED the doses have ranged from about 2.5 to about 100 mg once a day (QD). The PAH approved doses are generally slightly higher on a total mg/kg/day basis than the lowest dose used in ED. While no arginase inhibitor has been studied in man, it is estimated that an oral dose of about 1 to about 1000 mg per day would be effective in treating endothelial disorders, preferably about 10 to about 250 mg per day. The synergistic combination of both a PDE5i and an arginase inhibitor would result in reduced dosages of each to achieve similar effects to that of either agent given singly. Thus, it is anticipated that a synergistic combination is comprised of a ratio of PDE5 inhibitor to arginase inhibitor of about 1:10 to about 20:1, preferably from about 1:1 to about 10:1.

TABLE 1
PDE5 Inhibitors and Approved Dosages in Man.

| | Erectile Dysfunction | PAH |
|---|---|---|
| sildenafil | 50-100 mg QD | 20 mg TID |
| Tadalafil | 5-20 mg as needed | 40 mg QD |
| Vardenafil | 2.5-20 mg QD | 5 mg QD 4 weeks, |
| | | 5 mg BID* Clinical Trial Dose |

As used herein, the term “pharmaceutically-acceptable carrier” means a chemical composition with which at least one arginase inhibitor and at least one PDE inhibitor can be combined and which, following the combination, can be used to administer at least one arginase inhibitor and at least one PDE inhibitor to a patient.

As used herein, the term “physiologically-acceptable” ester or salt means an ester or salt form of the active ingredient which is compatible with any other ingredients of the pharmaceutical composition, which is not deleterious to the subject to which the composition is to be administered.

“Patient” refers to animals, preferably mammals, more preferably humans.

“Transurethral” or “intraurethral” refers to delivery of a drug into the urethra, such that the drug contacts and passes through the wall of the urethra and enters into the blood stream.

“Transdermal” refers to the delivery of a drug by passage through the skin and into the blood stream.

“Transmucosal” refers to delivery of a drug by passage of the drug through the mucosal tissue and into the blood stream.

“Penetration enhancement” or “permeation enhancement” refers to an increase in the permeability of the skin or mucosal tissue to a selected pharmacologically active agent such that the rate at which the drug permeates through the skin or mucosal tissue is increased.

“Carriers” or “vehicles” refers to carrier materials suitable for drug administration and include any such material known in the art such as, for example, any liquid, gel, solvent, liquid diluent, solubilizer, combinations thereof and the like, which is non-toxic and which does not interact with any components of the composition in a deleterious manner.

The term “sexual dysfunction” generally includes any sexual dysfunction in a patient, including an animal, preferably a mammal, more preferably a human. The patient can be male or female. Sexual dysfunctions can include, for example, sexual desire disorders, sexual arousal disorders, orgasmic disorders, sexual pain disorders, combinations thereof and the like. Female sexual dysfunction refers to any female sexual dysfunction including, for example, sexual desire disorders, sexual arousal dysfunctions, orgasmic dysfunctions, sexual pain disorders, dyspareunia, vaginismus, combinations thereof and the like. The female can be pre-menopausal or menopausal. Male sexual dysfunction refers to any male sexual dysfunctions including, for example, male erectile dysfunction and impotence.

The terms “treatment,” “treating,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and can include: inhibiting the disease or condition, i.e., arresting its development; and relieving the disease, i.e., causing regression of the disease.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically-acceptable diluent, carrier or vehicle. The specifications for the unit dosage forms for use in the present invention depend on the particular compound employed and the effect to be achieved, the pharmacodynamics associated with each compound in the host, and the like.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001).

It is to be understood that this application is not limited to particular embodiments described. It is also to be understood that the terminology used herein is for the purpose of describing particular embodiments only, and is not intended to be limiting, since the scope of the present application will be limited only by the appended claims.

Unless defined otherwise, all technical and scientific terms used herein have the same meaning as commonly understood by one of ordinary skill in the art. Although any methods and materials similar or equivalent to those described herein can also be used in the practice or testing of exemplary embodiments, the preferred methods and materials are now described. All publications mentioned herein are incorporated herein by reference in their entireties to disclose and describe the methods and/or materials in connection with which the publications are cited.

The singular forms “a,” “and,” and “the” include plural referents unless the context clearly dictates otherwise. Thus, for example, reference to “an arginine inhibitor” includes a plurality of such inhibitor compounds and reference to “the arginase” includes reference to one or more arginase polypeptides and equivalents thereof known to those skilled in the art, and so forth.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the recitation of a numerical range for a variable is intended to convey that the variable can be equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed. All references disclosed herein are incorporated by reference in their entirety.

### Arginase and Arginase Inhibitors

L-Arginine (Arg) is a conditionally essential amino acid, naturally found in dietary protein. It is converted to nitric oxide (NO) (Palmer et al. Nat Med 1987; 327:524-526; Moncada et al. N Engl J Med 1993; 329:2002-2012; Kam et al. Anaesthesia 1994; 49:515-521) and acts as a bronchodilator (Zoritch et al. Arch Dis Child 1995; 72:259-262; Gaston et al. Am J Respir Crit. Care Med 1994; 149:538-551) by a family of enzymes known as nitric oxide synthase (NOS). NO is an essential molecule that plays a role in a broad range of functions from vascular regulation, neurotransmission (Moncada et al. 1993, supra), host defense, and cytotoxicity (Nathan et al. Proc Natl Acad Sci 2000; 97:8841-8848) to physiologic control of airways (Gaston et al. 1994, supra). Under conditions of low L-arginine concentration, nitric oxide synthase is uncoupled and reduces oxygen (O₂) to superoxide (O₂) instead of generating nitric oxide (Xia et al. Proc Natl Acad Sci 1996; 93:6770-6774; Dias-Da-Motta et al. Brit J Haematol 1996; 93:333-340). Nitric oxide reacts rapidly with superoxide to form reactive nitric oxide species (RNOS) that could lead to worsening inflammation, oxidative stress and cellular damage (Demiryurek et al. Pharm Toxicology 1998; 82:113-117).

Expression of inducible NO synthase, the enzyme that catalyzes the production of NO from L-Arg, has been found in the epithelium of asthmatic patients but not in healthy non-asthmatic patients (Hamid et al. Lancet 1993; 342:1510-1513: Nijkamp et al. Arch Int Pharmoocodyn 1995; 329:81-96). Asthmatics have exhaled air NO levels that are 3.5 times higher than non-asthmatics, which are correlated with decrease in FEV₁ and are affected by therapy Kharitonov et al. Eur Respir J 1995; 8:295-7). Blocking of NO production by L-Arg analogues results in an increase in allergen-induced bronchoconstriction (Ricciardolo et al. Lancet 1996; 348:374-377). A deficiency of NO is involved in airway hyperreactivity (Meurs et al. Br J Pharmacol 1999; 126:559-562). Although asthma is clearly a multifactorial disease, there is some evidence that NO can play an important role in disease pathogenesis (Sanders et al. Am J Respir Cell Mol Biol 1999; 21:147-149). For reviews, see, e.g., Dweik Cleve Clin J. Med. June 2001; 68(6):486, 488, 490, 493; Gianetti et al. Eur J Clin Invest. August 2002; 32(8):628-35.

Arginase is an enzyme that catalyzes the hydrolysis of L-arginine to produce L-ornithine and urea, (Boucher et al. Cell Mol Life Sci 1999; 55:1015-1028). The enzyme is known to serve three important functions: production of urea, production of ornithine, and regulation of substrate arginine levels for nitric oxide synthase (Jenkinson et al., 1996, Comp. Biochem. Physiol. 114B:107-132; Kanyo et al., 1996, Nature 383:554-557; Christianson, 1997, Prog. Biophys. Molec. biol. 67:217-252). Urea production provides a mechanism to excrete nitrogen in the form of a highly soluble, non-toxic compound, thus avoiding the potentially dangerous consequences of high ammonia levels. L-ornithine is a precursor for the biosynthesis of polyamines, spermine, and spermidine, which have important roles in cell proliferation and differentiation. Arginase modulates production of nitric oxide by regulating the levels of arginine present within tissues.

In the presence of nitric oxide synthase (NOS), arginine is converted to nitric oxide (NO) and citrulline (Moncada et al. 1993, supra). The expression of arginase can be induced by a variety of cytokines involved in the inflammatory process (Solomons et al. Pediatr 1972; 49:933), particularly the Th2 cytokines. (Mori et al. 2000. Relationship between arginase activity and nitric oxide production. In L. Ignarro, editor. Nitric Oxide. Biology and Pathology. Academic Press, San Diego. 199-208.). Arginase regulates NO synthase activity by affecting the amount of L-arginine available for oxidation catalyzed by NO synthase activity. Thus, inhibition of arginase activity can enhance NO synthase activity, thereby enhancing NO-dependent smooth muscle relaxation in the corpus cavemosum and enhancing penile erection.

Arginase shares L-arginine as a common substrate with nitric oxide synthase (NOS). Elevated arginase restricts the supply of L-arginine NOS can use, restricting the production of nitric oxide (NO) and consequently cGMP. Since both NO synthase and arginase compete for the same substrate, the possibility of reciprocal regulation of both arginine metabolic pathways has been explored (Modolell et al., 1995, Eur. J. Immunol. 25:1101-1104; Wang et al., 1995, Biochem. Biophys. Res. Commun. 210:1009-1016). Furthermore, N_{ω}-hydroxy-L-arginine (L-HO-Arg), an intermediate in the NO synthase reaction (Pufahl et al., 1992, Biochemistry 31:6822-6828; Klau et al, 1993, J. Biol. Chem. 268:14781-14787; Furchgom, 1995, Annu. Rev. Pharmacol. Toxicol., 35:1-27; Yamaguchi et al., 1992, Eur. J. Biochem., 204:547-552; Pufahl et al., 1995, Biochemistry 34:1930-1941), is an endogenous arginase inhibitor (Chenais et al., 1993, Biochem. Biophys. Res. Commun., 196:1558-1565; Buga et al., 1996, Am. J. Physiol. Heart Circ. Physiol. 271:H1988-H1998 Daghigh et al., 1994, Biochem. Biophys. Res. Commun, 202; 174-180; Boucher et al., 1994, Biochem. Biophys. Res. Commun. 203:1614-1621). The phenomenon of reciprocal regulation between arginase and NO synthase has been examined (Chakder and Rattan, 1997, J. Pharmacol. Exp. Ther. 282:378-384; Langle et al., 1997, Transplantation 63:1225-1233; Langle et al., 1995, Transplantation 59:1542-1549). In the internal anal sphincter (IAS), it was shown that exogenous administration of arginase attenuates NO synthase-mediated non-adrenergic and non-cholinergic (NANC) nerve-mediated relaxation (Chakder and Rattan, 1997, J. Pharmacol. Exp. Ther. 282:378-384).

An excess of arginase has recently been associated with a number of pathological conditions that include gastric cancer (Wu et al., 1992, Life Sci. 51:1355-1361; Leu and Wang, 1992, Cancer 70:733-736; Straus et al., 1992, Clin. Chim. Acta 210:5-12; Ikemoto et al, 1993, Clin. Chem. 39:794-799; Wu et al., 1994, Dig. Dis. Sci. 39:1107-1112), certain forms of liver injury (Ikemoto et al., 1993, Clin. Chem. 39:794-799), and pulmonary hypertension following the orthotopic liver transplantation (Langle et al., 1997, Transplantation 63:1225-1233; Langle et al., 1995, Transplantation 59:1542-1549). Furthermore, high levels of arginase can cause impairment in NANC-mediated relaxation of the IAS (Chakder and Rattan, 1997, J. Pharmacol. Esp. Ther. 282:378-384). Previous studies have demonstrated that arginase pre-treatment causes significant suppression of the NANC nerve-mediated relaxation of the IAS (Chakder and Rattan 1997, J. Pharmacol. Exp. Ther. 282:378-384) that is mediated primarily via the L-arginine-NO synthase pathway (Rattan and Chakder, 1992, Am. J. Physiol. Gastrointest. Liver Physiol. 262: G107-G112; Rattan and Chakder, 1992, Gastroenterology 103:43-50). Impairment in NANC relaxation by excess arginase can be related to L-arginine depletion (Wang et al., 1995, Eur. J. Immunol. 25:1101-1104). Furthermore, suppressed relaxation could be restored by the arginase inhibitor L-HO-Arg. It is possible, therefore, that patients with certain conditions associated with an increase in arginase activity can stand to benefit from treatment with arginase inhibitors. However, an arginase inhibitor such as L-OH-Arg can not be selective since it also serves as a NO synthase substrate (Pufahl et al., 1992, Biochemistry 31:6822-6828; Furchgott, 1995, Annu. Rev. Pharmacol. Toxicol. 25:1-27; Pufahl et al, 1995, Biochemistry 34:1930-1941; Chemais et al., 1993, Biochem. Biophys. Res. Commun. 196:1558-1565; Boucher et al., 1994, Biochem. Biophys. Res. Commun. 203:1614-1621; Griffith and Stuehr, 1995, Annu. Rev. Physiol. 57:707-736). Because of this, the exact role of arginase in pathophysiology and the potential therapeutic actions of arginase inhibitors remains undetermined.

Arginase controls the metabolism of arginine into ornithine, which in turn gives rise to proline and polyamines (Mori et al. 2000, supra; Morris Annu Rev Nutr 2002; 22:87-105; Morris 2000. Regulation of arginine availability and its impact on NO synthesis. Nitric Oxide. Biology and Pathobiology. Academic Press, San Diego. 187-197; Mori et al. Biochem Biophys Res Commun 2000; 275:715-719). These downstream products of arginase activity can play a significant role in the pathogenesis of asthma, pulmonary hypertension and other inflammatory conditions, since proline is involved in collagen formation (Kershenobich et al. J Clin Invest 1970; 49:2246-2249; Albina et al. J Surg Res 1993; 55:97-102) and lung fibrosis (Endo et al. Am J Physiol Lung Cell Mol Physiol 2003; 285:L313-L321), processes that occur in airway wall thickening and airway remodeling (Tanaka et al. Inflamm Res 2001; 50:616-624: Elias et al. J Clin Invest 1999; 104:1001-1006; Elias et al. J Clin Invest 2003; 111:291-297; Busse et al. N Engl J Med 2001; 344:350-362).

Arginase Inhibitors

Two isozymes of arginase exist in most mammals. Arginase I functions in the urea cycle and is located primarily in the cytoplasm of the liver. Arginase II, which is involved in the regulation of the arginine/ornithine concentrations in the cell and can be found in the absence of other urea cycle enzymes. Arginase consists of three tetramers and requires a two-molecule metal cluster of manganese in order to maintain proper function. These Mn²⁺ ions coordinate with water, orientating and stabilizing the molecule and allowing water to act as a nucleophile and attack L-arginine, hydrolyzing it into ornithene and urea. A limited number of arginase inhibitors are known. These include: 2(S)-Amino-6-boronohexanoic acid (ABH), S-(2-boronoethyl)-L-cysteine (BEC), N^{ω}-hydroxy-nor-L-arginine (nor-NOHA), and N^{ω}-hydroxy-L-arginine (NOHA).

An increase in arginase activity has been associated with the pathophysiology of a number of conditions including endothelial disorders, including asthma, cardiovascular disorders, erectile dysfunction, female sexual dysfunction, inflammation, intermittent claudication, peripheral arterial occlusive disorders, pulmonary hypertension, Raynaud's disease, stroke and systemic hypertension. The use of an arginase inhibitor for the treatment of asthma is shown in numerous patents, such as U.S. Pat. Nos. 6,930,113, 6,462,044 and 6,331,543. The use of an arginase inhibitor for the treatment of erectile dysfunction, pulmonary hypertension and systemic hypertension is shown in U.S. Pat. No. 6,387,890. The use of an arginase inhibitor for the treatment of female sexual dysfunction is shown in U.S. Pat. No. 6,762,202. The use of an arginase inhibitor for the treatment of stroke is shown in several patents, such as U.S. Pat. Nos. 6,930,113, 6,462,044 and 6,331,543.

Thus, exemplary embodiments include methods of treating these conditions using a synergistically effective amount of an arginase inhibitor in combination with a PDE1, PDE2 and/or PDE5 inhibitor.

### Phosphodiesterase (PDE) Inhibitors

A phosphodiesterase is an enzyme that breaks a phosphodiester bond. There are 11 families of phosphodiesterases, named PDE1-PDE11, in mammals. The classification of these enzymes is based on their: amino acid sequences; substrate specificities; regulatory properties; pharmacological properties and tissue distribution. PDE enzymes are often targets for pharmacological inhibition due to their unique tissue distribution, structural properties, and functional properties. (Jeon Y, Heo Y, Kim C, Hyun Y, Lee T, Ro S, Cho J (2005). “Phosphodiesterase: overview of protein structures, potential therapeutic applications and recent progress in drug development”. Cell Mol Life Sci 62 (11): 1198-220.) Inhibitors of PDE can prolong or enhance the effects of physiological processes mediated by cAMP or cGMP by inhibition of their degradation by PDE.

The use of phosphodiesterase inhibitors for the treatment and prevention of diseases induced by the increased metabolism of cyclic guanosine 3′,5′-mono-phosphate (cGMP), such as hypertension, pulmonary hypertension, congestive heart failure, renal failure, myocardial infraction, stable, unstable and variant (Prinzmetal) angina, atherosclerosis, cardiac edema, renal insufficiency, nephrotic edema, hepatic edema, stroke, asthma, bronchitis, chronic obstructive pulmonary disease (COPD), cystic fibrosis, dementia, immunodeficiency, premature labor, dysmenorrhea, benign prostatic hyperplasis (BPH), bladder outlet obstruction, incontinence, conditions of reduced blood vessel patency, e.g., postpercutaneous transluminal coronary angioplasty (post-PTCA), peripheral vascular disease, allergic rhinitis, and glucoma, and diseases characterized by disorders of gut motility, such as irritable bowel syndrome (IBS) have been previously described in, for example, U.S. Pat. Nos. 5,849,741 and 5,869,486, WO98/49166 and WO 97/03985, the disclosures of each of which are incorporated herein by reference in their entirety.

Phosphodiesterase (PDE) inhibitors, notably PDE5 inhibitors, have revolutionized the treatment of a wide variety of disorders in which cell signaling mediated by cyclic guanidine monophosphate (cGMP) is compromised. The most famous examples of their utility are the widely prescribed use of sildenafil (Viagra), vardenafil (Levitra) and tadalafil (Cialis) for the treatment of erectile dysfunction (ED). In the penis, PDE5 inhibitors increase intracellular levels of cGMP by hindering the hydrolytic activity of PDE5, thus maintaining the vasodilator activity of cGMP. Specifically, inhibition of the hydrolysis of cGMP in the corpus cavernosum increases corporal smooth muscle relaxation and prolongs penile erection.

Although PDE5 inhibitors are effective and popular for the treatment of mild to moderate ED, there is a large population of patients with severe ED who respond poorly, if at all, to PDE5 inhibition, commonly when the ED is a consequence of having diabetes. Diabetic patients can have significant impairments in nitric oxide (NO)-bioavailability in the diabetic penile vasculature, thus corporeal cGMP levels are reduced and PDE5 inhibitor therapy is less efficacious in this ED patient population. The significant impairment in NO-bioavailability is a consequence of elevated arginase in diabetic corpus cavernosum. (Bivalacqua et al., 2004, Biochem. Biophys. Res. Commun., 283:923-927). Combining PDE5 inhibitors and arginase inhibitors synergistically enhances the benefits of each, enabling treatment for previously untreatable patients.

PDE5 inhibitors are gaining increasing acceptance as a treatment option for pulmonary arterial hypertension (PAH). Combining PDE5 inhibition with inhaled nitric oxide (NO) demonstrates benefits over using either approach in isolation in a variety of animal models of PAH, indicating that there is a shortage of NO. Arginase II has been shown to be elevated in pulmonary biopsies from patients with PAH. (Xu et al., 2004, FASEB Journal 18:1746). The level of serum arginase I is elevated in patients with hemolytic disorders such as sickle cell disease, a known cause of PAH. (Morris et al, 2005, J. Am. Med. Assoc., 294:81-90). Arginase II activity is also elevated in the vasculature of rats treated with monocrotaline, a well recognized model of PAH. PAH is a medical condition with unmet needs, which is treated with combination therapies which each incrementally improving the lives of the patients. Combining arginase inhibitors and PDE5 inhibitors that act synergistically together will result in significant benefits over using either treatment alone.

PDE5 inhibition is ineffective in the absence of sufficient cGMP produced as a consequence of NO signaling. However, one of the major contra-indications to the use of PDE5 inhibitors is patients who are talking nitrates because administration of high doses of systemic nitrates leads to systemic hypotension through overproduction of cGMP and the downstream signaling Inhaled NO is not useful in combination with PDE5 inhibitors because NO is only acting at the site of effect (pulmonary circulation) and systemic effects are not seen. In one of the key novel aspects of this invention is that inhibiting arginase acts in the same organ/spatially confined manner as inhibiting endothelial PDEs. The dramatic effect of arginase inhibition is really only seen in pathophysiologic states in which arginase is activated or up-regulated. Thus arginase inhibition and synergy will only really enhance NO production and thus produce synergy is states in which arginase is activated, at the sites in which arginase is elevated. This site specificity and spatial confinement leads to increases in cGMP production only in tissues where its production is pathologically depressed due to over activity of arginase.

The use of a phosphodiesterase inhibitor for the treatment of asthma is shown in numerous patents, such as U.S. Pat. Nos. 6,569,890, 6,218,400, and 6,087,368.

The use of a phosphodiesterase inhibitor for the treatment of erectile dysfunction is shown in numerous patents, such as U.S. Pat. Nos. 7,393,825, 7,235,625, and 6,218,400. The use of a phosphodiesterase inhibitor for the treatment of female sexual dysfunction is shown in numerous patents, such as U.S. Pat. Nos. 7,393,825 and 6,423,683.

The use of a phosphodiesterase inhibitor for the treatment of pulmonary hypertension is shown in U.S. Pat. Nos. 6,462,047 and 6,218,400.

The use of a phosphodiesterase inhibitor for the treatment of Raynaud's disease is shown in U.S. Pat. Nos. 6,423,683 and 6,165,975. The use of a phosphodiesterase inhibitor for the treatment of stroke and hypertension is shown in U.S. Pat. No. 6,218,400.

A combination of arginase inhibitors with inhibitors of phosphodiesterase PDE1, PDE2 and/or PDE5 and the use of such a combination for the treatment of endothelial disorders, including asthma, cardiovascular disorders, erectile dysfunction, female sexual dysfunction, inflammation, intermittent claudication, peripheral arterial occlusive disorders, pulmonary hypertension, Raynaud's disease, stroke, systemic hypertension, combinations thereof and the like are provided.

In an embodiment, a composition comprises a mixture of a synergistically-effective amount of at least one arginase inhibitor and at least one inhibitor of phosphodiesterase PDE1, PDE2 and/or PDE5.

Nature of the Arginase Inhibitors

A variety of arginase inhibitors can be adapted for use in exemplary compositions. The arginase inhibitor can be a reversible or irreversible arginase inhibitor, or an arginase antibody. Preferably, the arginase inhibitor is compatible for use, or can be adapted so as to be compatible for use, in a pharmaceutically-acceptable formulation or in a nutraceutical. Examples of suitable arginase inhibitors include, but are not necessarily limited to: 2(S)-Amino-6-boronohexanoic acid (ABH), S-(2-boronoethyl)-L-cysteine (BEC), N^{ω}-hydroxy-nor-L-arginine (nor-NOHA) and N^{ω}-hydroxy-L-arginine (NOHA), S-(+)-Amino-6-iodoacetamidohexanoic acid; S-(+)-Amino-5-iodoacetamidopentanoic acid; L-norvaline, combinations thereof, and the like. The arginase inhibitors used in exemplary embodiments can also include chemically-modified arginase inhibitors which are structurally modified to provide an additional source of NO, or another arginase inhibitor upon being degraded or metabolized in a patient. The arginase inhibitors used in exemplary embodiments can also include chemically-modified arginase inhibitors which are structurally modified to target delivery of the inhibitor to the desired site(s) of action. Arginase inhibitors used in exemplary embodiments can also include “prodrugs” of arginase inhibitors that are metabolized or degraded into arginase inhibitors. The arginase inhibitors can also include chemically-modified arginase inhibitors which are structurally modified to target delivery of the inhibitor to the desired site(s) of action where they are metabolized or degraded at the target site into arginase inhibitors.

Nature of the Phosphodiesterase Inhibitors

A variety of phosphodiesterase inhibitors can be adapted for use in the present invention. The phosphodiesterase inhibitors are inhibitors of phosphodiesterase PDE1, PDE2 and/or PDE5. The phosphodiesterase inhibitor can be a reversible or irreversible phosphodiesterase inhibitor, or a phosphodiesterase antibody. Preferably the phosphodiesterase inhibitor is compatible for use, or can be adapted so as to be compatible for use, in a pharmaceutically acceptable formulation or in a nutraceutical. Examples of suitable PDE1 inhibitors include: 5E3623 (Eisai), BAY 383045 (Bayer), HFV 1017 (Daiichi Fine Chemical), KF 19514 (Kyowa Hakko) and SCH 51866 (Schering-Plough). PDE2 inhibitors claimed include: BAY 607550 (Bayer). PDE5 inhibitors claimed include: Mirodenafil (SK Chemicals), Sildenafil (Pfizer), Tadalafil (Eli Lilly), Udenafil (Dong-A Pharmaceutical), Vardenafil (Bayer), Avanafil (Mitsubishi Tanabe Corp), Dasantafil (Schering-Plough), NM 702 (Nissan Chemical Industries), SLX 101 (Surface Logix) and UK 369003 (Pfizer). Other non-specific PDE inhibitors claimed include: Amlexanox (Takeda), Caffeine citrate (Mead Johnson), Doxofylline (ABC), Levosimendan (Orion), Mopidamol (Boehringer Ingelheim Pharma KG), Pentoxifylline (sanofi-aventis), Pemobendan (Boehringer Ingelheim Pharma KG), Propentofylline (sanofi-aventis), Vesnarinone (Otsuka Pharmaceutical), Ibudilast (Avigen), combinations thereof and the like.

In a particular embodiment, compositions can include at least one arginase inhibitor and at least one PDE1, PDE2 and/or PDE5 phosphodiesterase inhibitor combined in a single pharmaceutically-acceptable medium.

In another embodiment, at least one arginase inhibitor and at least one PDE1, PDE2 and/or PDE5 phosphodiesterase inhibitor are initially present in separate pharmaceutically-acceptable mediums, which can be combined at least one of before, during and after administration to an individual subject in need thereof to form a single pharmaceutically-acceptable medium that is administered to the patient.

The use of a combination of a synergistically-effective amount of at least one arginase inhibitor with at least one phosphodiesterase PDE1, PDE2 and/or PDE5 inhibitor for the treatment of endothelial disorders, including asthma, cardiovascular disorders, erectile dysfunction, female sexual dysfunction, inflammation, intermittent claudication, peripheral arterial occlusive disorders, pulmonary hypertension, Raynaud's disease, stroke, systemic hypertension, combinations thereof and the like is also provided.

Arginase shares L-arginine as a common substrate with nitric oxide synthase (NOS). Elevated arginase restricts the supply of L-arginine NOS can use, restricting the production of nitric oxide (NO) and consequently cGMP. PDE1, PDE2 and PDE5 regulate cGMP in both the heart and vasculature. Inhibitors of PDEs, particular PDE5 inhibitors, are used in the treatment of a variety disorders in which NO signaling is impaired. These disorders include erectile dysfunction and pulmonary arterial hypertension. PDE inhibitors are generally less effective in treating conditions where arginase activity is elevated. The combined use of arginase inhibitors and PDE1, PDE2 and PDE5 inhibitors has synergistic benefits in such conditions.

### Cardiovascular Modulation by Nitric Oxide (NO) and Regulation by Phosphodiesterases

Cardiovascular modulation by nitric oxide (NO) can be divided into two primary mechanisms. One depends upon NO activation of soluble guanylate cyclase (sGC) and the subsequent generation of cyclic guanosine monophosphate (cGMP), while the other is cGMP-independent and involves protein S-nitrosylation or nitration (reviewed in (Bian, et al., 2006 J. Pharmacol. Sci. 101: 271-279; Hess, et al., 2005 Nat. Rev. Mol. Cell. Biol. 6: 150-166). To a great extent, the balance between these pathways depends upon redox status that influences net NO chemistry and NO and cGMP synthetic capacity by NOS and sGC, respectively (Zimmet, et al., 2006 Circulation 114: 1531-1544)(Zimmet and Hare, 2006). Once synthesized, cGMP regulates cellular function by binding to allosteric sites in cyclic nucleotide phosphodiesterases influencing their activity and by stimulating protein kinase G (PKG, also cGK) (Hofmann, et al., 2006 Physiol. Rev. 86: 1-23). By its phosphorylation of channels, receptors, kinases, and phosphatases (Lincoln, et al., 2001 J. Appl. Physiol. 91: 1421-1430), PKG serves as a primary modulator of vascular tone, and plays a key role in cell survival, endothelial permeability, and vascular homeostasis and proliferation. In the heart, PKG regulates contractile function (Hofmann, et al., 2006 Physiol. Rev. 86: 1-23), and serves as a brake to counter both acute and chronic stress responses and cardiac remodeling (Takimoto, et al. 2005 Circ. Res. 96: 100-109; Takimoto, et al., 2005 Nat. Med. 11: 214-222).

The importance of cGMP to NO signaling has naturally led to research on the catabolic enzymes that control its fate once synthesized. These proteins are members of the 21-gene family of phosphodiesterases which have been grouped into 11 different primary isoenzymes (with a total of 48 isoforms) based on substrate affinity, selectivity, and regulation mechanisms (Table 1). Of these enzymes, PDE5, PDE6, and PDE9 are highly selective for cGMP, PDE1, PDE2, and PDE11 have dual substrate affinity, and PDE3 and PDE10 are cGMPsensitive but cAMP-selective. In the cardiovascular system, the primary cGMP-PDEs with known activity are PDE1, PDE2, and PDE5. PDE1 is a Ca²⁺/calmodulin dependent enzyme, PDE2, a cGMP-stimulated cAMP esterase that can also hydrolyze cGMP, and PDE5 the first identified selective cGMP esterase. An additional cGMP-selective PDE9A was recently identified (Wang, et al., 2003 Gene 314: 15-27), with an isoform (PDE9A5) expressed at low levels in heart, though its role if any remains unknown.

TABLE 2
Family of PDEs, their substrate specificity and tissue expression,
adapted from Kass, et al., 2007 Cardiovasc. Res. 75: 303-314

| PDE | | |
|---|---|---|
| Isoenzyme | Substrate | Tissue Expression |
| 1 | Ca²⁺/calmodulin- | Heart, brain, lung, smooth muscle |
| | stimulated | |
| 2 | cGMP-stimulated | Adrenal gland, heart, lung, liver, |
| | | platelets |
| 3 | cGMP-inhibited | Heart, lung, liver, platelets, adipose |
| | | tissue, inflammatory cells |
| 4 | cAMP-selective | Sertoli cells, kidney, brain, liver, lung, |
| | | inflammatory cells |
| 5 | cGMP-specific | Lung, platelets, vascular smooth |
| | | muscle, heart |
| 6 | cGMP-specific | Photoreceptor |
| 7 | cAMP-specific, | Skeletal muscle, heart, kidney, brain, |
| | high affinity | pancreas, T lymphocytes |
| 8 | cAMP-selective | Testes, eye, liver, skeletal muscle, |
| | | heart, kidney, ovary, brain, T |
| | | lymphocytes |
| 9 | cGMP-specific | Kidney, liver, lung, brain, possibly |
| | | heart |
| 10 | cGNO-sensitive, | Testes, brain |
| | cAMP-selective | |
| 11 | cGMP-sensitive, | Skeletal muscle, prostate, kidney, liver, |
| | dual specficity | pituitary and salivary glands, testes |

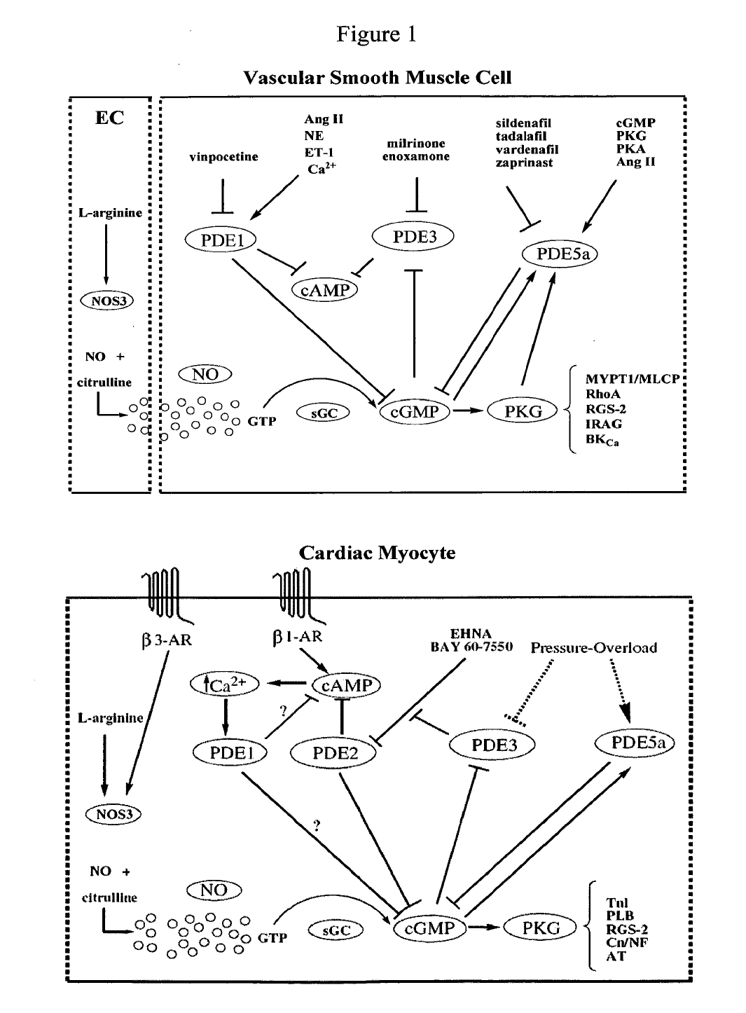

cGMP can inhibit PDE3, a cAMP esterase expressed in heart and vascular tissue. Among these PDEs, PDE5 has been best studied due to the existence of highly selective inhibitors, and remains the only one of the family of PDEs for which targeted inhibitors are FDA approved to treat a chronic clinical disease—erectile dysfunction and more recently pulmonary hypertension. FIG. 1 summarizes the roles of PDEs in vascular smooth muscle or cardiac myocytes. PDEs modulate acute stimulation cascades but can also be up-regulated in chronic conditions that can result in proliferative remodeling and desensitization to cGMP signaling. Small molecule inhibitors and signal activators for each PDE are shown at the top. Smooth muscle cell: NOS3-derived NO diffuses from neighboring endothelial cells (EC) interacting with soluble guanylate cyclase (cGC) to convert GTP to cGMP. cGMP can be hydrolyzed by PDE1 in the presence of Ca²⁺/calmodulin (Ca/CM) stimulation, and can also hydrolyze cAMP. PDE3 hydrolyzes primarily cAMP, but this can be inhibited competitively by cGMP. PDE5 selectively hydrolyzes cGMP, and cGMP and its distal effecter kinase-protein kinase G (PKG) also activate the enzyme. Activation of PKG results in phosphorylation of myosin light chain phosphatases (MLCP), RhoA, regulator of g-protein signaling (RGS-2), inositol 1,4,5-trisphosphate receptor-associated PKG substrate; IRAG, and calcium-sensitive potassium channels (BKca) that serve to reduce smooth muscle tone. Cardiac myocyte: NO possibly derived from several intracellular NOS isoforms (eNOS being coupled to β3 adrenergic receptors) stimulates cGMP synthesis. PDE regulation is similar to that in vascular smooth muscle, with the addition of PDE2, which increases cAMP hydrolysis when stimulated by cGMP, but can also hydrolyze cGMP. Agonists and small molecule inhibitors would be similar for those shown in the upper panel. Chronic stimulation by pressure over-load lowers PDE3 but increases PDE5 activity to alter the balance of cAMP/cGMP regulation (From Kass et al., 2007 Cardiovasc. Res. 75: 303-314)

Modulation of Nitric Oxide—cGMP System by Phosphodiesterases in the Cardiovascular System

In the cardiovascular system, cGMP hydrolysis is thought to be accomplished by PDE1, PDE2, and PDE5. PDE1 contains an auto-inhibitory domain which maintains low activity in the absence of Ca²⁺, and neighboring calmodulin binding domains that restore full activation in the presence of Ca²⁺-calmodulin (Sonnenburg, et al., 1995 Biol. Chem. 270: 30989-31000). An intriguing feature of PDE1 is its activation by specific Ca²⁺ pools entering from the extracellular space (Goraya, et al., 2005 Cell Signal 17: 789-797) suggesting compartmentation, though this remains to be clarified in vascular smooth muscle or cardiomyocytes. PDE1 has three primary isoforms (a, b, c) that are all expressed in vascular smooth muscle. It is stimulated by norepinephrine, angiotensin II, and endothelin-1 by their elevation of intracellular calcium, and this serves to lower cGMP levels and augment vasoconstriction (Hagiwara, et al., 1984 Biochem. Pharmacol. 33: 453-457). Inhibition of PDE1 with vinpocetine, an often used but not very specific inhibitor, has little effect on basal cGMP or cAMP in pulmonary vascular tissue, but enhances NO-stimulated dilation suggesting an interaction with NO-derived cGMP (Evgenov, et al. 2006 Am. J. Physiol. Lung Cell Mol. Physiol. 290: L723-L729). Chronic up-regulation of PDE1 has been associated with nitrate tolerance (Kim, et al., 2001 Circulation 104: 2338-2343) and vascular proliferation (Nagel, et al. 2006 Circ. Res. 98: 777-784; Rybalkin, et al., 1997 J. Clin. Invest. 100: 2611-2621).

Although PDE1 is strongly expressed in the heart (Yanaka, et al., 2003 Biosci. Biotechnol. Biochem. 67: 973-979), its physiologic role and indeed even precise cell of origin remains unclear (Sonnenburg, et al., 1998 Methods 14: 3-19). Lack of genetic models targeting PDE1 as well as selective inhibitors has limited such research to date.

PDE2 is not a primary PDE in vascular smooth muscle, but is expressed in cardiac myocytes, and recent data supports its role in the targeted regulation of cGMP and cAMP. In rat myocytes, cGMP synthesis was assayed by a sarcolemmal membrane targeted olfactory cGMP-gated channel current (Castro, et al., 2006 Circulation 113: 2221-2228). The current was enhanced more by natriuretic peptide (NP) than NO donors, and PDE2 inhibition potentiated this current with both stimuli. However, the functional role of this modulation remains unknown, as PDE2 inhibition alone has little influence on resting myocyte contraction (Mongillo, et al. 2006 Circ. Res. 98: 226-234). It is also unclear if PDE2 preferentially catabolizes cGMP or cAMP (or both simultaneously), and under what conditions. Though PDE2 hydrolyzes cGMP when this is the primary substrate (Castro, et al., 2006 Circulation 113: 2221-2228), it appears to target cAMP when there is co-generation of cGMP and cAMP (Mongillo, et al. 2006 Circ. Res. 98: 226-234). The development of novel and selective PDE2 inhibitors, such as BAY 60-7550 (Castro, et al., 2006 Circulation 113: 2221-2228), and advances in fluorescent imaging methods can help resolve some of these questions.

PDE5 was first identified as a cGMP-binding protein in lung tissue, and only later was it revealed to have cGMP hydrolytic activity. It has since been shown to play a key role in vascular smooth muscle tone particularly in the venous system of the corpus cavernosum and the pulmonary vasculature. Protein expression and activity are also well documented in the cerebellum, stomach, small and large intestine, bladder, and platelets (Lin, et al., 2006 Curr. Pharm. Des. 12: 3439-3457). Early physiologic studies explored the role of PDE5 using the inhibitor zaprinast; which also has significant cross-reactivity with PDE1 as well. However, in the late 1980's, highly selective and potent PDE5 inhibitors such as sildenafil and tadalafil were developed, and this greatly improved our understanding of this PDE.

PDE5 appears to interact closely with NO-sGC generated cGMP as the effectiveness of PDE5 inhibitors are generally blocked by NOS inhibitors such as nitro-L-arginine methyl ester. While hypoxia-induced pulmonary hypertension is substantially ablated by sildenafil, the drug has little effect in mice lacking NOS3 (eNOS) (Zhao, et al., 2001 Circulation 104: 424-428). Pharmacologic inhibition of NOS suppresses the vasodilator effects of PDE5 in vitro (Shukla, et al., 2005 Eur. J. Pharmacol. 517: 224-231; Takagi, et al., 2001 Eur. J. Pharmacol. 411: 161-168) and in vivo (Weimann, et al., 2000 Anesthesiology 92: 1702-1712), and PDE5 inhibitors are less effective in disorders associated with reduced NOS activity, such as diabetes (Bivalacqua, et al., 2004 Int. J. Impot. Res. 16: 21-29). In experimental diabetic rats, the blunted improvement of erectile function by sildenafil is restored by eNOS gene transfer into the corpus cavernosum (Bivalacqua, et al., 2004 Int. J. Impot. Res. 16: 21-29). As already noted, auto-activation of PDE5 by cGMP generated by NO plays an important feedback role in NO signaling. For example, exogenous administration of NO to platelets or aortic tissue triggers an initial marked rise and then rapid decline in cellular cGMP, with the latter attributed to cGMP-PDE5 activation (Mullershausen, et al., 2001 J. Cell Biol. 155: 271-278). NO can also activate PDE5 by direct protein S-nitrosylation, though this remains to be verified.

### Insufficient Nitric Oxide Signaling in the Pathogenesis of Vasculopathy

Nitric oxide (NO) is the major endothelial signaling molecule responsible for mediating vasorelaxation (Durante, 2001 Cell Biochem. Biophys. 35: 19-34; Loscalzo, et al., 1995 Prog. Cardiovasc. Dis. 38: 87-104). Nitric oxide is produced by nitric oxide synthase (NOS), for which L-arginine is the exclusive substrate (Palmer, et al., 1988 Nature 333: 664-666). The constitutive forms of this enzyme, neuronal NOS (nNOS; NOS1) and endothelial NOS (eNOS; NOS3) are the principal NOS isoforms involved in the induction of vasorelaxation. NO synthesized in non-adrenergic, non-cholinergic (NANC) nerves by nNOS, and by arterial endothelial cells via eNOS, diffuses to the underlying vascular smooth muscle cells where it activates relaxation by elevating intracellular levels of cGMP.

Decreased NO is known to be involved in the pathogenesis of a number of vascular disorders, including hypertension, atherosclerosis, and diabetic vasculopathy (John, et al., 2003 Curr. Hypertens. Rep. 5: 199-207; Soriano, et al., 2001 J. Mol. Med. 79: 437-448). Studies have demonstrated an increased abundance of eNOS in human aortic endothelial cells cultured in the presence of excess glucose, and in arteries obtained from diabetic animals (Cosentino, et al., 1997 Circulation 96: 25-28). This suggests that decreased eNOS is not responsible for endothelial NO deficiency in diabetes, which seems likely, instead, to result from a decrease in the availability of the eNOS substrate, L-arginine (Cooke, et al., 1992 J. Clin. Invest. 90: 1168-1172; Drexler, et al., 1991 Lancet 338: 1546-1550; Tousoulis, et al., 2002 Vasc. Med. 7: 203-211).

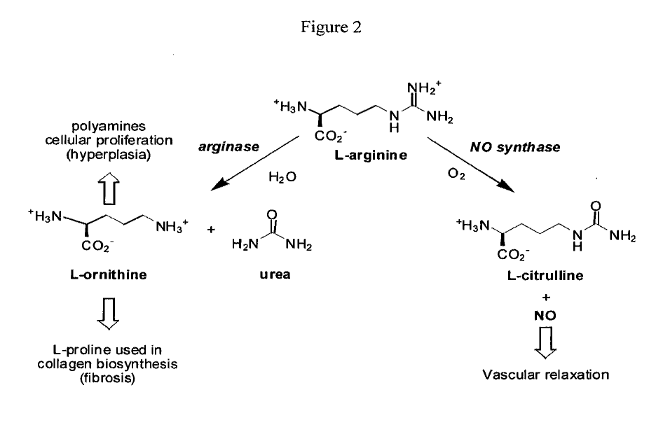

The decreased availability of L-arginine in a wide variety of vasculopathies can be the result of its increased catabolism by arginase (Simon, et al., 2003 Circ. Res. 93: 813-820). Arginase is expressed in a number of extrahepatic tissues, including blood vessels (Bachetti, et al., 2004 J. Mol. Cell. Cardiol. 37: 515-523; Durante, et al., 1997 J. Biol. Chem. 272: 30154-30159; Wei, et al., 2000 Am. J. Physiol. Cell Physiol. 279: C248-C256). Arginase competes with eNOS for L-arginine, which it uses as a substrate for urea production. Therefore, the relative activities and concentrations of arginase and eNOS in endothelial cells are reciprocal determinants of the production of either urea or NO, as shown in FIG. 2. NO, the product of eNOS activity is the principal mediator of vasorelaxation. Depletion of L-arginine by arginase, which is elevated in diabetic vasculopathy, results in decreased NO production and increased production of L-ornithine and reactive oxygen species leading to vascular stiffness and damage. This suggests that dysregulation of arginase expression or activity can be involved in the development of diabetic vascular lesions.

### Arginase is Becoming a Validated Molecular Target for Treating Vascular Endothelial Dysfunction

Arginase is a 105 kD homotrimeric enzyme that requires manganese for the hydrolysis of L-arginine to form L-ornithine and urea. Two genetically distinct isozymes, arginase I and arginase II, have evolved with differing tissue distributions and subcellular locations in mammals. The vast majority of arginase activity in the body is due to cytosolic arginase I which is found predominantly in the liver, where it catalyzes the final cytosolic step of the urea cycle and is responsible for the generation of approximately 10 kg of urea per year by the average human adult. Arginase II is a mitochondrial enzyme that does not appear to function in the urea cycle and is more widely distributed in numerous tissues, for example, kidney, brain, skeletal muscle, and liver.

In a number of vascular diseases in which endothelial dysfunction are a significant component, arginase up-regulation or activation has been suggested as the proximate cause (Bivalacqua, et al., 2001 Biochem. Biophys. Res. Commun. 283: 923-927; Demougeot, et al., 2007 Life Sci. 80: 1128-1134; Johnson, et al., 2005 Am. J Physiol. Regul. Integr. Comp. Physiol. 288: R1057-R1062; Marinova, et al., 2008 J. Pharmacol. Sci. 106: 385-393; Morris, 2006 Treat Respir. Med. 5: 31-45; Romero, et al., 2008 Circ. Res. 102: 95-102; Santhanam, et al. 2007 Circ. Res. 101: 692-702; White, et al., 2006 Hypertension 47: 245-251; Xu, et al. 2004 FASEB J. 18: 1746-1748; Zhang, et al., 2001 FASEB J. 15: 1264-1266). Although the affinity of eNOS for L-arginine is much higher than that of arginase, the activity of arginase can be more than 100-fold that of NOS, suggesting that it is competitive in utilizing L-arginine (Wu, et al., 1998 Biochem. J. 336: 1-17). Competitive depletion of L-arginine in endothelial cells leads to vascular lesions via three inter-related mechanisms: 1) decreased production of NO, the critical endothelial signal for vasorelaxation, 2) increased production of urea and ornithine by arginase, leading to vascular hyperplasia and fibrosis (Endo, et al., 2003 Am. J. Physiol. Lung Cell Mol. Physiol. 285: L313-L321; Keskinege, et al., 2001 Cancer Detect Prev. 25: 76-79), and 3) destabilization of dimeric eNOS, leading to increased production of reactive oxygen species (ROS) including superoxide and hydrogen peroxide (Xia, et al., 1996 Proc. Natl. Acad. Sci. U.S.A. 93: 6770-6774; Xia, et al., 1997 Proc. Natl. Acad. Sci. U.S.A. 94: 6954-6958), causing direct endothelial damage (Bivalacqua, et al., 2003 J. Urol. 169: 1911-1917; Bivalacqua, et al., 2005 Sex Med. 2: 187-197; disc. 197-188; Rabelink, et al., 2006 Arterioscler. Thromb. Vasc. Biol. 26: 267-271). Inhibition of arginase is associated with increased NO production in endothelial cells (Chicoine, et al., 2004 Am. J. Physiol. Lung Cell Mol. Physiol. 287: L60-L68; Santhanam, et al. 2007 Circ. Res. 101: 692-702).

### Arginase and PDE5A are Expressed Together in the Endothelium

Without being bound by any particular theory, PDE5 and arginase are both believed to be present in the endothelium, with PDE5 being present in the caveoli. PDE5 inhibition results in increased NOS activity. It is believed that combining PDE5 inhibition and arginase inhibition is synergistic in decreasing ROS, in more ways than by simply providing more arginine to make NO to make cGMP.

### Small Molecule Arginase Inhibitors as a Therapeutic for Vasculopathy

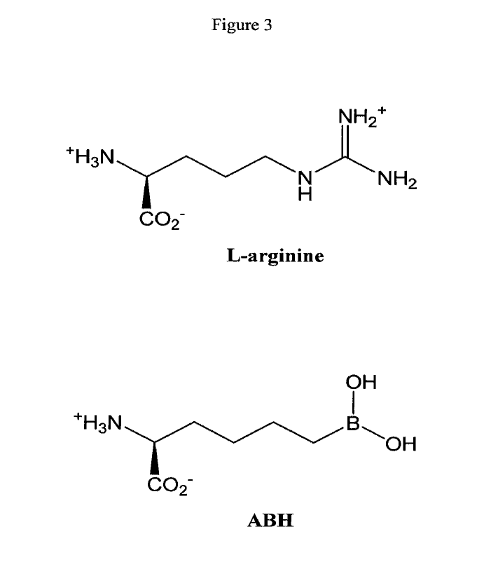

A number of inhibitors of arginase have been described (Reviewed in (Christianson, 2005 Acc. Chem. Res. 38: 191-201)), however, none have been developed as therapeutic agents for use in alleviating vascular dysfunction in patients. In part, this has been because the inhibitory affect of many have failed to be reproduced in tissues. The only published study in which in vivo arginase inhibition is demonstrated shows inhibition of serum arginase with N^{ω}-hydroxy-nor-L-arginine (nor-NOHA), but not of endothelial arginase, due to poor bioavailability of nor-NOHA (Reid, et al., 2007 Am. J. Physiol. Gastrointest. Liver Physiol. 292: G512-G517). Studies show that a small molecule inhibitor of arginase, 2(S)-Amino-6-boronohexanoic acid (ABH) has high potential as a therapeutic for a number of vasculopathies supported by considerable in vivo efficacy (see Examples). The structure of ABH closely resembles the natural substrate L-arginine but contains a boronic acid group, as seen in FIG. 3.

ABH was first designed and synthesized in the lab of Dr David Christianson, based on the crystal structure of the rat Arginase I molecule (Baggio, et al., 1997 J. Am. Chem. Soc. 119: 8107-8108). Arginase is a 105 kD homotrimeric metalloenzyme that contains a binuclear manganese cluster in the active site of each subunit. The binuclear manganese cluster is required for maximal hydrolysis of L-arginine to form L-ornithine and urea (Kanyo, et al., 1996 Nature 383: 554-557). This structure provided a basis for guiding the design and synthesis of nonreactive arginine analogs that could act as possible enzyme inhibitors, or antagonists of arginase. Based on the crystal structure of the ternary arginase-ornithine-borate complex, it was postulated that the boronic acid analog of L-arginine, ABH, would bind avidly to arginase as the hydrated anion. Boronic acids are effective aminopeptidase and serine protease inhibitors because they bind as tetrahedral transition state analogs. The electron-deficient boron atom of a boronic acid invites the addition of a suitable nucleophile (e.g. a protein-bound nucleophile or a solvent molecule) to yield a stable, anionic tetrahedral species (Cox, et al., 1999 Nat. Struct. Biol. 6: 1043-1047).

ABH has been demonstrated to be the most potent inhibitor of either arginase I or arginase II (reviewed in (Christianson, 2005 Acc. Chem. Res. 38: 191-201). However, ABH, unlike many other arginase inhibitors that fail to effectively decrease arginase activity in vivo, exhibits promising activity in vivo (Baggio, et al., 1999 J. Pharmacol. Exp. Ther. 290: 1409-1416; Kim, et al., 2004 J. Nutr. 134: 2873S-2879S; disc. 2895S; Ryoo, et al., 2008 Circ. Res. 102: 923-932). This suggests its high potential as a drug for treating endothelial dysfunction, such as occurs in diabetic vasculopathy.

### The Effects of Diabetes on Cardiovascular Health

There are 20.8 million children and adults in the United States, or 7% of the population, who have diabetes mellitus. Only approximately 5-10% of the diagnosed cases of diabetes among Americans are due to a failure to produce insulin (type 1 diabetes). The vast majority of diabetics have type 2 diabetes (originally called “adult onset”), which results from insulin resistance combined with relative insulin deficiency. As a result of the aging population, and increases in obesity and sedentary lifestyle, the incidence of diabetes is climbing, with 789,000 new cases diagnosed annually. Vascular disease remains the most significant complication of diabetes, and is central to the development of a number of diabetic pathologies. Diabetes-associated damage to the small blood vessels (microvascular disease) leads to retinopathy (the most common cause of blindness among non-elderly adults in the U.S.), neuropathy, and nephropathy. Damage to the larger vessels (macrovascular disease) can result in coronary artery disease (leading to angina and myocardial infarction), stroke, peripheral vascular disease, and erectile dysfunction. Despite management, more than 65% of people with diabetes die from heart disease or stroke (http://www.cdc.gov/diabetes/pubs/pdf/ndfs_{—}2005.pdf: “National Diabetes Fact Sheet”).

Several complex mechanisms are thought to contribute to the pathogenesis of diabetic vasculopathy, including excessive protein glycosylation on vascular endothelial cells (Wautier, et al., 2004 Circ. Res. 95: 233-238), and accelerated atherosclerosis resulting from insulin resistance-mediated dyslipidemia (Nigro, et al., 2006 Endocr. Rev. 27: 242-259). However, a diminished capability for endothelium-dependent vasorelaxation, resulting in chronically increased vascular tone or “stiffness”, is a pathological characteristic that is common to animal models of diabetes, and human diabetes (Luscher, et al., 1997 Clin. Cardiol. 20: II-3-10). Recent understanding of the molecular mechanisms contributing to vascular stiffness in diabetes has revealed new potential molecular targets for treating diabetic vasculopathy.

ED is one of the earliest symptoms of diabetic vasculopathy. It occurs in 50% of men with type 1 or type 2 diabetes (Rendell, et al., 1999 JAMA 281: 421-426; Saenz de Tejada, et al., 1989 N. Engl. J. Med. 320: 1025-1030), and the rate of sexual dysfunction is only slightly lower in diabetic women (Bultrini, et al., 2004 Sex Med. 1: 337-340). ED develops early in diabetic men, typically within 10 years of the onset of diabetes, and its occurrence is strongly predictive of more widespread diabetic vascular disorders, such as coronary heart disease and peripheral atherosclerosis (Haffner, et al., 1998 N Engl. J. Med. 339: 229-234; Kannel, et al., 1979 Circulation 59: 8-13). It has been suggested that ED can represent the first sign of undiagnosed diabetes in as many as 10% of ED patients (Sairam, et al., 2001 BJU Int. 88: 68-71).

Patients with Diabetic ED Respond Poorly to PDE5 Inhibitors Because they have Compromised NO/cGMP Signaling as a Result of Elevated Arginase

The NO/cGMP signaling cascade has been well established as the main functional control system for penile corporal smooth muscle relaxation and penile erection. It has been demonstrated that arginase activity and expression is elevated in the corpus cavernosum of diabetic humans and animals (Bivalacqua, et al., 2001 Biochem. Biophys. Res. Commun. 283: 923-927; Jelodar, et al., 2007 J. Reprod. Dev. 53: 317-321). Diabetic ED can therefore be an important model for generalized diabetic vasculopathy. One indication that NO signaling is compromised in diabetes is the fact that PDE5 inhibitor therapy (such as sildenafil) is less efficacious in patients with diabetes-induced ED when compared to patients with other causes of ED (Rendell, et al., 1999 JAMA 281: 421-426). Inhibition of arginase I in tissue extracts from diabetic corpus cavernosa with ABH have been shown to increase levels of cGMP (Bivalacqua, et al., 2001 Biochem. Biophys. Res. Commun. 283: 923-927).

In the penis, PDE5 inhibitors increase intracellular levels of cGMP by hindering the hydrolytic activity of PDE5, thus maintaining the vasodilator activity of cGMP. Sildenafil inhibits the hydrolysis of cGMP in the corpus cavernosum, thereby increasing corporal smooth muscle relaxation and prolonging penile erection. However, there are significant impairments in NO-bioavailability in the diabetic penile vasculature, thus corporeal cGMP levels are reduced and PDE5 inhibitor therapy is less efficacious in this ED patient population.

### Pulmonary Arterial Hypertension (PAH)

Pulmonary arterial hypertension (PAH) is a chronic, progressive disease characterized by increased pulmonary vascular resistance of the lung microvasculature, intimal hyperplasia and smooth muscle cell hypertrophy, and in situ thrombosis (Rubin, 2006 Proc. Am. Thorac. Soc. 3: 111-115). PAH disease progression leads to right heart failure and death (D'Alonzo, et al., 1991 Ann. Intern. Med. 115: 343-349; Vlahakes, et al., 1981 Circulation 63: 87-95). PAH is defined by mean pulmonary arterial pressure that exceeds 25 mm Hg at rest or 30 mm Hg during exercise, with mean pulmonary-capillary wedge pressure or left ventricular end diastolic pressure ≦15 mm Hg and pulmonary vascular resistance greater than 3 Wood units (Barst et al., 2004 Am. Coll. Cardiol. 43: 40S-47S). Unfortunately and despite significant efforts to diagnose patients earlier in the disease process, the disease is most often diagnosed months or years after symptoms first appear. As a consequence, the majority of patients present with advanced disease and marked functional impairment (Hoeper, 2005 Drugs 65: 1337-1354).

### The Role of the Nitric Oxide Signaling Pathway in PAH

In the lungs, NO is a potent pulmonary vasodilator and inhibitor of platelet activation and vascular smooth muscle cell proliferation. Decreased levels of eNOS have been reported in pulmonary vascular tissue in patients with PAH (Giaid, et al., 1995 N. Engl. J. Med. 333: 214-221). As mentioned earlier the effects of NO are mediated via cyclic guanosine monophosphate (cGMP) in vascular smooth muscle cells. The intracellular concentration of cGMP is regulated by phosphodiesterases, which rapidly degrade cGMP in vivo (Ahn, et al., 1991, Adv. Exp. Med. Biol. 308: 191-197; Beavo et al., 1990 Trends Pharmacol. Sci. 11: 150-155). Phosphodiesterase 5 (PDE5) is highly expressed in the lung, and its expression is increased in PAH (Braner, et al., 1993 Am. J. Physiol. 264: H252-258). Therefore, drugs that selectively inhibit PDE5 can prolong endogenous NO signaling and prove efficacious in PAH. Sildenafil is a PDE5 inhibitor that was originally approved for erectile dysfunction and was recently approved for the treatment of PAH (Rubin, et al., 2005 Ann. Intern. Med. 143: 282-292; Weimann, et al., 2000 Anesthesiology 92: 1702-1712). In the SUPER-1 trial, three times daily sildenafil improved exercise capacity (assessed by the 6-minute walk test), WHO functional class, and decreased pulmonary arterial pressure and pulmonary vascular resistance (Galie, et al. 2005 N. Engl. J. Med. 353: 2148-2157). In 2009, tadalafil, another PDE5 inhibitor, was approved for the treatment of PAH (marketed as Adcirca TM).

### PAH and Elevated Arginase Activity

One of the most commonly used models for PAH is the rat monocrotaline (MCT) model where MCT causes pathologic alterations in the lung and heart similar to human PAH. In a detailed study of the MCT rat PAH model both in arginase II protein and activity were increased in pulmonary arterial endothelial cells from rats twenty-four days after MCT treatment (Sasaki, et al., 2007 Am. J. Physiol. Lung Cell Mol. Physiol. 292: L1480-L1487). This is in agreement with an earlier study by Xu et al., demonstrating abnormally high levels of expression of arginase II in the pulmonary endothelial cells in biopsies from patients with PAH by immunostaining (Xu, et al. 2004 FASEB J. 18: 1746-1748). Further, in the same study, pulmonary artery endothelial cells derived from PAH lung had higher arginase II expression and produced lower NO than control cells in vitro.

PAH is also strongly associated with a variety of chronic hereditary and acquired hemolytic anemias including sickle cell disease (SCD), thalassemia intermedia, paroxysmal nocturnal hemoglobinuria, hereditary spherocytosis and stomatocytosis, microangiopathic hemolytic anemias and pyruvate kinase deficiency (Rother, et al., 2005 JAMA 293: 1653-1662).

The intravascular hemolysis in all these conditions causes erythrocytes to release arginase I and hemoglobin into the circulation: elevated circulating arginase has been clearly demonstrated for both SCD and thalassemia. In addition to this it has been shown that arginase I expression is elevated in erythrocytes of patients with SCD and thalessemia prior to hemolysis. As damaging as the massive release of arginase I into the circulation is the impact on the levels of nitric oxide in circulation is compounded by the release of hemoglobin. The hemoglobin released overwhelms the homeostatic mechanisms in place to remove it. This cell-free hemoglobin then acts as a nitric oxide scavenger (Morris, 2006 Treat Respir. Med. 5: 31-45; Morris, et al., 2005 JAMA 294: 81-90; Morris, et al., 2005 Ann. N.Y. Acad. Sci. 1054: 481-485).

PDE5 Inhibitors in Combination with Inhaled Nitric Oxide Lead to Better Treatment of PAH than Either in Isolation

Because the vasodilator effects of NO are largely mediated via cGMP-dependent mechanisms (Ichinose, et al., 2004 Circulation 109: 3106-3111; Schlossmann, et al., 2003 Ann. Med. 35: 21-27), it has been hypothesized that inhibition of the cGMP-metabolizing phosphodiesterases (PDEs) would augment the ability of inhaled NO to dilate the pulmonary vasculature by further increasing cGMP levels in pulmonary vascular smooth muscle cells (Humbert, et al. 2004 J. Am. Coll. Cardiol. 43: 13S-24S. In the lungs, at least six cGMP-metabolizing PDE families (PDE 1, 2, 3, 5, 9, and 10) have been identified (Fujishige, et al., 1999 J. Biol. Chem. 274: 18438-18445; Phillips, et al., 2005 Am. J. Physiol. Lung Cell Mol. Physiol. 288: L103-L115; Rabe, et al., 1994 Am. J. Physiol. 266: L536-L543; Schlossmann, et al., 2003 Ann. Med. 35: 21-27; Sharma, et al., 1986 J. Biol. Chem. 261: 14160-14166; Soderling, et al., 1998 J. Biol. Chem. 273: 15553-15558). Zaprinast, an inhibitor of several cGMP-metabolizing PDEs, potentiates and markedly prolongs pulmonary vasodilation induced by inhaled NO when administered in lambs with chemically induced PAH (Ichinose, et al., 1998 Anesthesiology 88: 410-416; Ichinose, et al., 1995 J. Appl. Physiol. 78: 1288-1295). Subsequently, oral administration of the more potent, clinically approved PDE5 inhibitor sildenafil has been shown to produce selective pulmonary vasodilation in experimental models, as well as in patients with PAH (Lepore, et al., 2002 Am. J. Cardiol. 90: 677-680; Michelakis, et al., 2002 Circulation 105: 2398-2403; Michelakis, et al., 2003 Circulation 108: 2066-2069; Weimann, et al., 2000 Anesthesiology 92: 1702-1712). In addition, sildenafil augmented the pulmonary vasodilator response to inhaled NO, when administered in an aerosolized form in lambs with PAH (Ichinose, et al., 2001 Crit. Care Med. 29: 1000-1005), suggesting an important modulatory role of PDE5 on pulmonary vascular tone. However, the specific roles of other cGMP-metabolizing PDEs in modulating the pulmonary vasodilator response to inhaled NO remain largely unexplored.

Exemplary embodiments also encompass the use of pharmaceutical compositions of an arginase inhibitor and a PDE inhibitor to practice the exemplary method using compositions comprising arginase inhibitor and a PDE inhibitor and a pharmaceutically-acceptable carrier. Arginase inhibitors and PDE inhibitors, or other active agents for administration according to exemplary embodiment can be formulated in a variety of ways suitable for administration according to exemplary methods. In general, these compounds are provided in the same or separate formulations in combination with a pharmaceutically-acceptable excipient(s).

In an embodiment, a synergistically-effective amount of at least one arginase inhibitor and at least one phosphodiesterase (PDE) inhibitor is used in the preparation of a medicament for the treatment of an endothelial disorder.

In another embodiment, a synergistically-effective amount of at least one arginase inhibitor and at least one phosphodiesterase (PDE) inhibitor is used in the preparation of a medicament for the treatment of an endothelial disorder where the endothelial disorder is asthma, a cardiovascular disorder, erectile dysfunction, female sexual dysfunction, inflammation, intermittent claudication, a peripheral arterial occlusive disorder, pulmonary hypertension, Raynaud's disease, stroke, systemic hypertension or a combination thereof.

Pharmaceutical compositions that are useful in exemplary methods can be administered systemically in oral solid formulations, ophthalmic, suppository, aerosol, topical, other similar formulations, combinations thereof and the like. In addition to an arginase inhibitor and a PDE inhibitor, such pharmaceutical compositions can contain pharmaceutically-acceptable carriers and other ingredients known to enhance and facilitate drug administration. Other possible formulations, such as nanoparticles, liposomes, resealed erythrocytes, and immunologically based systems can also be used to administer an arginase inhibitor and a PDE inhibitor according to the methods of the invention.

The preparation and use of pharmaceutical compositions comprising a compound useful for treatment of the diseases disclosed herein as an active ingredient is provided. Such a pharmaceutical composition consists of the active ingredients alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically-acceptable ester or salt, such as in combination with a physiologically-acceptable cation or anion.

Exemplary compounds and compositions can be formulated as pharmaceutically-acceptable neutral or acid salt forms. Pharmaceutically-acceptable salts include, for example, those formed with free amino groups such as those derived from hydrochloric, hydrobromic, hydroiodide, phosphoric, sulfuric, acetic, citric, benzoic, fumaric, glutamic, lactic, malic, maleic, succinic, tartaric, p-toluenesulfonic, methanesulfonic acids, gluconic acid, combinations thereof and the like, and those formed with free carboxyl groups, such as those derived from sodium, potassium, ammonium, calcium, ferric hydroxides, isopropylamine, triethylamine, 2-ethylamino ethanol, histidine, procaine, combinations thereof, and the like.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of exemplary pharmaceutical compositions described herein is contemplated include, but are not limited to, humans and other primates, mammals including commercially relevant mammals such as cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in exemplary methods can be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A suitable pharmaceutical composition can be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically-acceptable carrier, and any additional ingredients in a pharmaceutical composition of the invention will vary, depending upon the identity, size, and condition of the subject treated and further depending upon the route by which the composition is to be administered. By way of example, the composition can comprise between about 0.001% and about 50% (w/w) of each of the active ingredients, preferably between about 0.01% and about 25% (w/w) of each of the active ingredients, and more preferably between about 0.1% and about 10% (w/w) of each of the active ingredients.

In addition to the active ingredient, a pharmaceutical composition of the invention can further comprise one or more additional pharmaceutically active agents. Particularly contemplated additional agents include anti-emetics and scavengers such as cyanide and cyanate scavengers.

The formulations can optionally further include a preservative. Suitable preservatives include, but are not limited to, a benzyl alcohol, phenol, chlorobutanol, benzalkonium chloride, combinations thereof, and the like. The formulation can be stored at temperatures of about 4° C. to improve storage stability. Formulations can also be lyophilized, in which case they generally include cryoprotectants such as sucrose, trehalose, lactose, maltose, mannitol, combinations thereof, and the like. Lyophilized formulations can be stored over extended periods of time, even at ambient temperatures.

Controlled- or sustained-release formulations of a pharmaceutical composition of the invention can be made using conventional technology.

A formulation of an exemplary pharmaceutical composition suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Solid dosage forms for oral administration can include capsules, tablets, effervescent tablets, chewable tablets, pills, powders, sachets, granules, gels, combinations thereof, and the like. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the present invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin, combinations thereof, and the like. Tablets and pills can be prepared with enteric coatings.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, lubricating agents, combinations thereof, and the like. Known dispersing agents include, but are not limited to, potato starch, sodium starch glycollate, combinations thereof, and the like. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, sodium phosphate combinations thereof, and the like. Known granulating and disintegrating agents include, but are not limited to, corn starch, alginic acid, combinations thereof, and the like. Known binding agents include, but are not limited to, gelatin, acacia, pre-gelatinized maize starch, polyvinylpyrrolidone, hydroxypropyl methylcellulose, combinations thereof, and the like. Known lubricating agents include, but are not limited to, magnesium stearate, stearic acid, silica, talc combinations thereof, and the like.

Tablets can be non-coated or they can be coated using known methods to achieve delayed disintegration in the gastrointestinal tract of a subject, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin.

Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Liquid formulations of a pharmaceutical composition of a combination of a synergistically effective amount of at least one arginase inhibitor and at least one phosphodiesterase PDE1, PDE2 and/or PDE5 inhibitor which are suitable for oral administration can be prepared, packaged, and sold either in liquid form or in the form of a dry product intended for reconstitution with water or another suitable vehicle prior to use.

Liquid suspensions can be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils, such as liquid paraffin, combinations thereof, and the like. Liquid suspensions can further comprise one or more additional ingredients including, but not limited to, suspending agents, dispersing or wetting agents, emulsifying agents, demulcents, preservatives, buffers, salts, flavorings, coloring agents, and sweetening agents. Oily suspensions can further comprise a thickening agent. Suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose and hydroxypropylmethylcellulose. Dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Emulsifying agents include, but are not limited to, lecithin and acacia. Preservatives include, but are not limited to, methyl, ethyl, or n-propyl-para-hydroxybenzoates, ascorbic acid, and sorbic acid. Sweetening agents include, for example, glycerol, propylene glycol, sorbitol, sucrose, and saccharin. Thickening agents for oily suspensions include, for example, beeswax, hard paraffin, and cetyl alcohol.

Liquid solutions of the active ingredient in aqueous or oily solvents can be prepared in substantially the same manner as liquid suspensions, the primary difference being that the active ingredient is dissolved, rather than suspended in the solvent. Liquid solutions of the pharmaceutical composition of the invention can comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention can be prepared using known methods. Such formulations can be administered directly to a subject, used, for example, to form tablets, to fill capsules, or to prepare an aqueous or oily suspension or solution by addition of an aqueous or oily vehicle thereto. Each of these formulations can further comprise one or more of dispersing or wetting agent, a suspending agent, and a preservative. Additional excipients, such as fillers and sweetening, flavoring, or coloring agents, can also be included in these formulations.

A suitable pharmaceutical composition can also be prepared, packaged, or sold in the form of oil-in-water emulsion or a water-in-oil emulsion. The oily phase can be a vegetable oil such as olive or arachis oil, a mineral oil such as liquid paraffin, or a combination of these. Such compositions can further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate.

These emulsions can also contain additional ingredients including, for example, sweetening or flavoring agents.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations can, for example, comprise from about 0.001% to about 10% (w/w) of each of the active ingredients, preferably about 0.01% to about 5% (w/w) of each of the active ingredients, and more preferably about 0.05% to about 1% (w/w) of each of the active ingredients, although the concentration of the active ingredient can be as high as the solubility limit of the active ingredient in the solvent. Formulations for topical administration can further comprise one or more of the additional ingredients described herein.

Dosage forms for topical administration of the compounds and compositions of the present invention can include creams, sprays, lotions, gels, ointments, coatings for condoms and the like. Administration of the cream or gel can be accompanied by use of an applicator or by transurethral drug delivery using a syringe with or without a needle or penile or vaginal insert or device, and is within the skill of the art. Typically a lubricant and/or a local anesthetic for desensitization can also be included in the formulation or provided for use as needed. Lubricants include, for example, K-Y jelly (available from Johnson & Johnson) or a lidocaine jelly, such as Xylocalne 2% jelly (available from Astra Pharmaceutical Products). Local anesthetics include, for example, novocaine, procaine, tetracaine, benzocaine and the like.

Topical administration can also involve transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Examples of suitable carriers include, for example, water, silicone, waxes, petroleum jelly, polyethylene glycol, propylene glycol, liposomes, sugars, and the like. The compositions can also include one or more permeation enhancers including, for example, dimethylsulfoxide (DMSO), dimethyl formamide (DMF), N,N-dimethylacetamide (DMA), decylmethylsulfoxide (C10MSO), polyethylene glycol monolaurate (PEGML), glyceral monolaurate, lecithin, 1-substituted azacycloheptan-2-ones, alcohols, combinations thereof, and the like.

A pharmaceutical composition of the invention can be prepared, packaged, or sold in a formulation suitable for rectal administration. Such a composition can be in the form of, for example, a suppository, a retention enema preparation, and a solution for rectal or colonic irrigation.

Suppository formulations can be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations can further comprise various additional ingredients including, but not limited to, antioxidants and preservatives.

Retention enema preparations or solutions for rectal or colonic irrigation can be made by combining the active ingredient with a pharmaceutically acceptable liquid carrier. As is well known in the art, enema preparations can be administered using, and can be packaged within, a delivery device adapted to the rectal anatomy of the subject. Enema preparations can further comprise various additional ingredients including, but not limited to, antioxidants and preservatives.

A pharmaceutical composition of the invention can be prepared, packaged, or sold in a formulation suitable for vaginal administration. Such a composition can be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation can be made by combining the active ingredient with a pharmaceutically acceptable liquid carrier. As is well known in the art, douche preparations can be administered using, and can be packaged within, a delivery device adapted to the vaginal anatomy of the subject. Douche preparations can further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations can be prepared, packaged, or sold in a form suitable for bolus administration or for continuous administration. Injectable formulations can be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations can further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

An exemplary pharmaceutical composition can be prepared, packaged, or sold in a formulation suitable for pulmonary administration via the buccal cavity. Such a formulation can comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant can be directed to disperse the powder or using a self-propelling solvent/powder-dispensing container such as a device comprising the active ingredient dissolved or suspended in a low-boiling propellant in a sealed container. Preferably, such powders comprise particles wherein at least about 98% of the particles by weight have a diameter greater than about 0.5 nanometers and at least about 95% of the particles by number have a diameter less than about 7 nanometers. More preferably, at least about 95% of the particles by weight have a diameter greater than about 1 nanometer and at least about 90% of the particles by number have a diameter less than about 6 nanometers. Dry powder compositions preferably include a solid fine powder diluent such as sugar and are conveniently provided in a unit dose form.

Low boiling propellants generally include liquid propellants having a boiling point of below about 65° F. at atmospheric pressure. Generally the propellant can constitute about 50 to about 99.9% (w/w) of the composition, preferably about 60 to about 99% (w/w) of the composition, and more preferably about 70 to about 95% (w/w) of the composition, and the active ingredient can constitute about 0.01 to about 20% (w/w) of the composition, preferably about 0.1 to about 10% (w/w) of the composition. The propellant can further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery can also provide the active ingredient in the form of droplets of a solution or suspension. Such formulations can be prepared, packaged, or sold as aqueous or dilute alcoholic solutions or suspensions, optionally sterile, comprising the active ingredient, and can conveniently be administered using any nebulization or atomization device. Such formulations can further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration can, for example, comprise from about as little as about 0.1% (w/w) and as much as about 100% (w/w) of the active ingredient, and can further comprise one or more of the additional ingredients described herein.

A pharmaceutical composition of the invention can be prepared, packaged, or sold in a formulation suitable for buccal administration. Such formulations can, for example, be in the form of tablets or lozenges made using conventional methods, and can, for example, about 0.01 to about 20% (w/w) about 0.01% to about 5% (w/w) of each of the active ingredients, preferably about 0.05 to about 10% (w/w) about 0.01% to about 5% (w/w) of each of the active ingredients, and most preferably about 0.1 to about 5% (w/w) about 0.01% to about 5% (w/w) of each of the active ingredients, the balance comprising an orally dissolvable or degradable composition and, optionally, one or more of the additional ingredients described herein. Alternately, formulations suitable for buccal administration can comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and can further comprise one or more of the additional ingredients described herein.

A pharmaceutical composition of the invention can be prepared, packaged, or sold in a formulation suitable for ophthalmic administration. Such formulations can, for example, be in the form of eye drops including, for example, an about 0.1—about 1.0% (w/w) solution or suspension of the active ingredient in an aqueous or oily liquid carrier. Such drops can further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials, combinations thereof, and the like. Other “additional ingredients” which can be included in the pharmaceutical compositions of the invention are known in the art.

Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc., which are incorporated herein by reference.

The compounds can be administered to an animal as frequently as several times daily, or it can be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

In some embodiments, the invention also includes a kit comprising a formulation comprising a unit dose of at least one arginase inhibitor, and at least one PDE inhibitor, or combination thereof, and a pharmaceutically acceptable excipient to administer the dosage form according to a desired regimen or exemplary regimen dependent upon the particular condition to be treated, patient age, patient weight, and the like. Kits can optionally include instructions for using the components of the kit to practice the subject methods. The instructions for practicing the subject methods are generally recorded on a suitable recording medium. For example, the instructions can be printed on a substrate, such as paper or plastic, etc. As such, the instructions can be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the interne, are provided. An example of this embodiment is a kit that includes a web address where the instructions can be viewed and/or from which the instructions can be downloaded. As with the instructions, this means for obtaining the instructions is recorded on a suitable substrate.

The inhibitors can be administered to a patient as frequently as several times daily, or it can be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disorder being treated, the type and age of the patient, etc.

The dosage regimen for treating a condition with the compounds and/or compositions of exemplary embodiments is selected in accordance with a variety of factors, including the type, age, weight, sex, diet and medical condition of the patient, the severity of the dysfunction, the route of administration, pharmacological considerations such as the activity, efficacy, pharmacokinetic and toxicology profiles of the particular compound used, whether a drug delivery system is used, and whether the compound is administered as part of a drug combination. Thus, the dosage regimen actually used can vary widely and therefore can deviate from the preferred dosage regimen set forth herein.

Particular embodiments are described below with reference to the following examples. These examples are provided for the purpose of illustration only and the application is not limited to these examples, but instead encompasses all variations which are evident as a result of the teaching provided herein.

### EXAMPLES

### Example 1

### ABH Improves Erectile Function in Aged Rodents

Old age, like diabetes, is known to be associated with endothelial dysfunction (such as peripheral vascular disease and erectile dysfunction). Arginase expression is consistently higher in aged, compared with younger animals, which is at least in part responsible for age-related vascular stiffness (Berkowitz, et al., 2003 Circulation 108: 2000-2006; Bivalacqua, et al., 2007 Am. J. Physiol. Heart Circ. Physiol. 292: H1340-1351). The effect of ABH in restoring age-related vascular function is demonstrated.

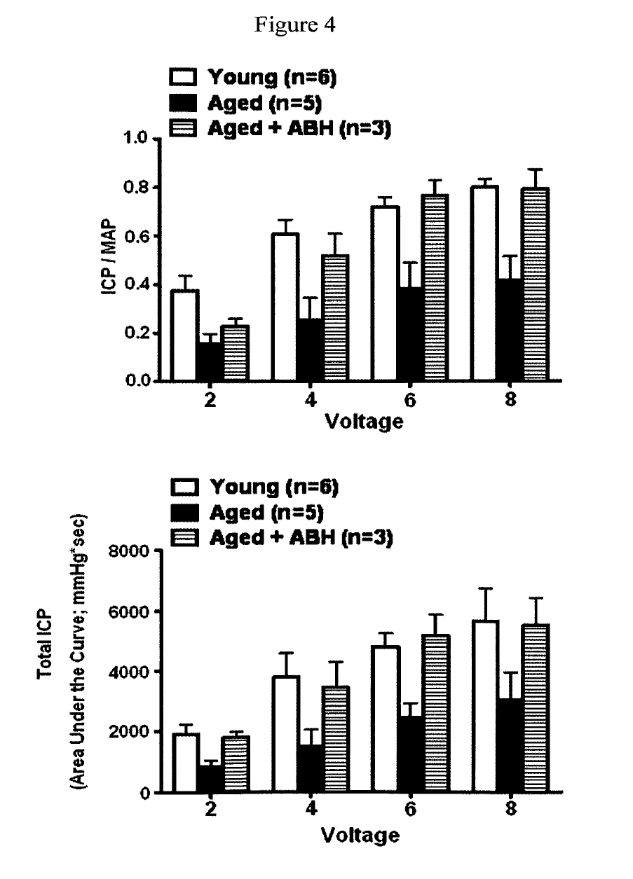

Aged (22-26 months) Fisher rats were given either ABH (6 mg/kg in their drinking water) or plain water for 28 days. Young (4-8 months) were given plain water for 28 days. Rats were then anesthetized and erectile function was evaluated as an indicator of vascular function, by measuring intra-cavernous pressure following electrical stimulation of the cavernous nerve using an electrode placed around the pelvic nerve which innervates the penis (Cama, et al., 2003 Biochemistry 42: 8445-8451). The change in intracavernosal pressure was measured via a catheter placed in the corpora cavernosa. A range of stimulation frequencies were used to measure relaxation effects (and consequentially vascular dependent erectile responses) over their entire functional range. A marked increase in the ratio of the mean peak intracavernous pressure (ICP) to mean arterial pressure (MAP) (FIG. 4A) as well as total ICP (FIG. 4B)(area under the erectile curve) was observed in ABH-treated rats compared with controls in response to cavernous nerve stimulation (CNS) in aged rats and aged rats treated with ABH (6 mg/kg) in the drinking water for 28 days. There is marked improvement in all penile hemodynamic parameters after CNS in rats treated with ABH. These data show that systemic administration of the arginase inhibitor ABH improves an indicator of vascular function, vasodilation in the penis, in aging rats.

### Example 2

### ABH Improves Systemic Vascular Stiffness in Aged Rodents

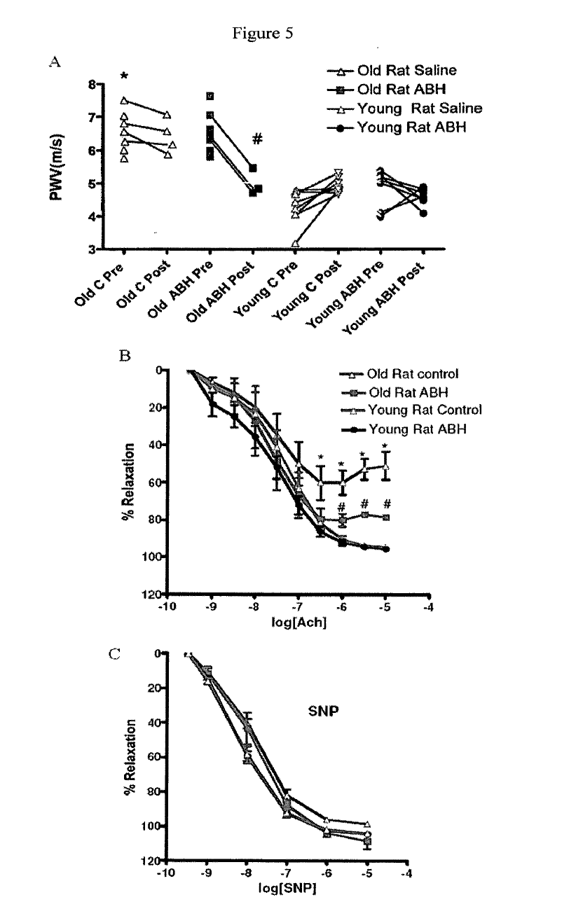

ABH, in addition to improving a localized indicator of vascular function, was shown to be effective in modifying systemic indicators of vascular relaxation/stiffness in aged rats as indicated by measuring the effects of ABH treatment on pulse wave velocity in aged rats. When the heart contracts it generates a pulse, or energy wave, that travels through the circulatory system. The speed of travel of this pulse wave (the pulse wave velocity or PWV) provides a systemic, non-invasive measure of arterial stiffness and hence vascular health. PWV was determined in aged (22-26 months) Fisher rats, treated with ABH as described in Example 1. Prior to treatment, aged rats exhibited significantly greater vascular stiffness than young (4-8 months) control rats. PWV was measured before and after 28 days of treatment with ABH (6 mg/kg) (See FIG. 5A). ABH improved vascular function in aortic rings tested in organ chambers (See FIG. 5B), while an endothelial-independent NO donor, SNP had no effect (C). *p<0.01 vs. young and #p<0.05 vs. old pre-treated, (n=3-8). Aged rats treated with ABH exhibited a marked decrease in vascular stiffness, with PWV measurements that were similar to those of young rats. This affect was not observed in untreated aged rats. There was a small but statistically significant increase in vascular stiffness in young control rats over the 28 days of the experiment. This change however was not observed in rats that were treated with ABH.

To investigate whether these changes in vascular stiffness could be related to specific measures of endothelial function, rats were euthanized following treatment, and the thoracic aortas dissected and placed in organ chambers as previously described (White, et al., 2006 Hypertension 47: 245-251). Rat aortic rings were pre-constricted using phenylephrine (1 μM), and vasorelaxant response to the endothelial-dependent (NO mediated) vasodilator acetylcholine (ACh), administered at 1 μM to 10 μM, was determined. Untreated aged rats showed significant impairment in ACh-responsiveness compared to young rats (FIG. 5B). However, following 28 days of oral treatment with ABH, vascular relaxation in aged rats was substantially improved. In contrast, responses to the endothelial-independent NO donor, SNP, were not affected by ABH treatment, indicating that the vasorelaxant effects of ABH are mediated via effects on endothelial cells. ABH had no effect on vascular endothelial relaxation in young rats.

### Example 3

### ABH Increases Nitric Oxide (NO) and Reduces Reactive Oxygen Species (ROS) in Aortas of Aged Rodents

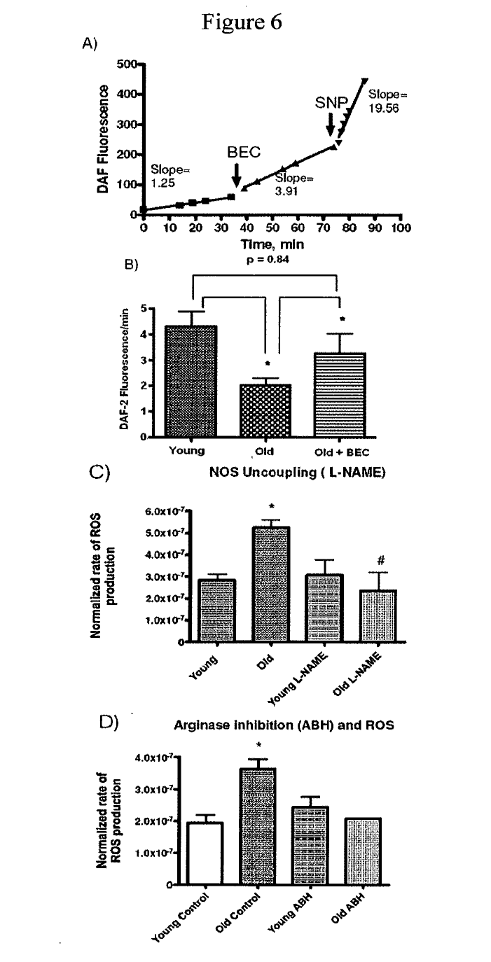

ABH improves endothelial function by inhibiting arginase and consequently increasing NO production (by increasing L-arginine availability to eNOS) and decreasing levels of ROS (by preventing eNOS uncoupling). This was demonstrated when levels of NO and ROS in aortas collected from young (4-8 months) and old (22-26 month) Fisher rats, with or without exposure to BEC, an ABH analog, were compared. Aortic rings were dissected and pinned down, endothelial side up, on a silastic coated culture dish, and then exposed to either the NO-sensitive dye DAF, or the O₂-sensitive dye DHE. Since the interaction of NO or ROS with its specific dye is cumulative, the rate of production of each species is proportional to the slope of the fluorescence emitted from the dye as measured using epifluorescence microscopy. Treatment of old rat aortic segments with the ABH analog BEC augments NO production, as measured by the slope of the DAF fluorescence (See FIG. 6A), and returns it to basal levels in young rats (P=0.004 for old vs. old+BEC; P=0.84 for old+BEC vs. young) (See FIG. 6B). SNP endothelial-independent NO donor was included as a control (Santhanam et al 2007). Reduction by exposure to nitro-L-arginine methyl ester, a NOS inhibitor, indicates that uncoupled NOS is the source of elevated ROS in aging rat aortic endothelium (See FIG. 6C) Inhibiting arginase by ABH exposure returns NOS to its coupled state, reducing ROS in old aorta to that observed in aortas collected from young rats (*p<0.01).

The addition of BEC increased the slope of DAF fluorescence in aged rat aorta, as did the addition of sodium nitrosprusside (SNP), an NO donor used as a positive control. This supports the hypothesis that arginase inhibition results in increased vascular NO production. A corollary of this effect is a decrease in ROS production by re-coupling eNOS to NO production. ROS concentrations in aortas collected from old rats were significantly greater than those in young rats. This resulted from deranged NOS activity (uncoupled NOS), since ROS levels could be reduced using the NOS-inhibitor nitro-L-arginine methyl ester (FIG. 6C). In aortas treated with ABH, ROS production was significantly reduced in aged aortas (FIG. 6D), demonstrating that by restoring the supply of L-arginine substrate, ABH reduces the production of damaging ROS by the uncoupled eNOS. Collectively, these data demonstrate that inhibition of arginase by ABH and its analogs has two therapeutically-important molecular effects: 1) restoration of NO production, and 2) reduction of ROS production. Similar techniques are used to examine whether ABH can restore NO production and decrease ROS production in vascular endothelial cells and corporal tissue from type 2 diabetic rats.

### Example 4

### Inhibition of Elevated Arginase Activity in Diabetic Human Vascular Explants with ABH

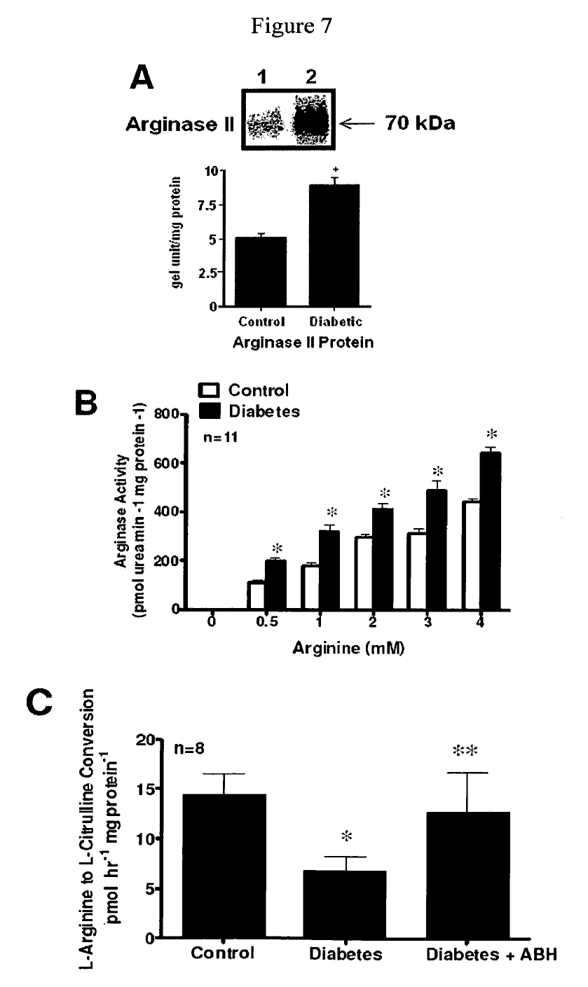

Having demonstrated the effectiveness of ABH in healthy and aged animal models, evidence was gathered showing that ABH might be effective in countering vasculopathies associated with diabetes. It is known that dysfunctional vascular endothelium of diabetic humans corpus cavernosum exhibits significant defects in the NO pathway (Rendell, et al., 1999 JAMA 281: 421-426; Saenz de Tejada, et al., 1989 N. Engl. J. Med. 320: 1025-1030). In order to study the role of arginase in the development of these defects, corpus cavernosum tissue extracts were obtained from non-diabetic and diabetic men undergoing placement of a penile prosthesis for the management of their severe ED (Bivalacqua, et al., 2001 Biochem. Biophys. Res. Commun. 283: 923-927). Using RT-PCR, arginase II expression was found to be significantly up-regulated in diabetic vs. control corpora cavernosum (data not shown). Densitometric analysis of western blots confirmed that arginase II protein is present in significantly greater amounts in corporal extracts taken from men with diabetes (See FIG. 7A, where arginase II (Arg 2) protein expression in human corpus cavernosum from control (lane 1) and diabetic (lane 2) demonstrates a significantly greater amount of arginase II protein in corporal tissue obtained from diabetic men (A), n=8-10; *P<0.05 compared to control.) Arginase activity is greater in human diabetic corporal tissue compared to controls as determined by urea production in the presence of a range of concentrations of labeled arginine (See FIG. 7B), *P<0.05 compared to control. Constitutive nitric oxide synthase activity (eNOS activity; calcium-dependent conversion of L-arginine to L-citrulline) is greater in human corporal tissue from diabetic patients vs. controls (See FIG. 7C), *P<0.05 when compare to controls; in the presence of the selective arginase inhibitor ABH, eNOS activity in human diabetic corpora caversosa is restored, **P<0.05 when compared to diabetic (Bivalacqua, et al., 2001 Biochem. Biophys. Res. Commun. 283: 923-927).

Diabetic corporal tissue extracts were prepared for the measurement of arginase activity by the production of urea in the presence of labeled arginine (FIG. 7B). Diabetic corporal tissues had significantly greater arginase activity compared to control tissue (p<0.05) over a range of arginine substrate concentrations. This supports the suggestion that increased arginase activity can be responsible for the significant reduction in eNOS activity in diabetic corporal tissue, as we have observed here compared with control corpus cavernosum. Importantly, when 1 mM ABH was added to diabetic penile extracts, eNOS activity improved to levels that were comparable to those of control corpus cavernosum (FIG. 7C). These data provide evidence that ABH has substantial potential to counter arginase-dependent mechanisms underlying the pathogenesis of diabetic endothelial dysfunction of the penile vascular bed.

### Example 5

### Inhibition of Arginase Restores Erectile Function in a Rodent Model of Type 1 Diabetes

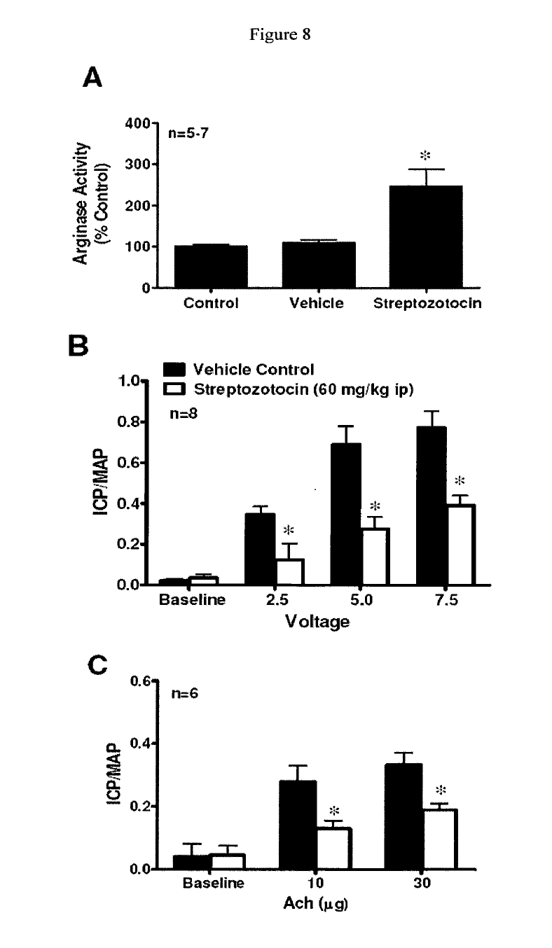

Rats were treated with streptozotocin (STZ; 60 mg/kg i.p.), a pancreatic beta cell toxin that produces a primary type 1 diabetic state. Markedly higher arginase activity was observed in diabetic rat penes 2 months after the induction of type 1 diabetes when compared to age-matched vehicle (citrate buffer) treated rats (FIG. 8A), *P<0.05 compared to control and vehicle. Elevated arginase activity was associated with impaired neurogenic-mediated (electrical stimulation, FIG. 8B) and endothelium-dependent (intra-cavernosal ACh injection, FIG. 8C) erectile responses in STZ-treated rats, *P<0.05 compared to vehicle control. These analyses were performed 2 months post treatment with STZ at which time penile eNOS activity is known to be significantly reduced in this model (Bivalacqua, et al., 2003 J. Urol. 169: 1911-1917). These data demonstrate the suitability of animal models of diabetes in the study of erectile dysfunction as a model for cardiovascular implications of arginase over-expression.

### Example 6

### Characterization of the Effectiveness of ABH in Improving Vascular Endothelial Function Ex Vivo in an Animal Model of Type 2 Diabetes

Biochemical and functional parameters of endothelial functions in a model of type 2 diabetes (Zucker Diabetic Fatty (ZDF) Rats) and in a control rat strain (Zucker Lean Control (ZLC) rat), are used to investigate the ex vivo effectiveness of ABH in improving these parameters. Vascular arginase activity and its role in the development of diabetic endothelial pathology have been previously characterized in models of Type 1 diabetes (Jelodar, et al., 2007 J. Reprod. Dev. 53: 317-321; Romero, et al., 2008 Circ. Res. 102: 95-102). However, although animal models of Type 1 diabetes are easy to produce (a number of methods are available to deplete pancreatic islet cells), they can not adequately recapitulate the vasculopathy of the 95% of diabetic people who suffer from the Type 2 form (insulin resistance). ZDF rats are a well characterized model of Type 2 diabetes, and have previously been demonstrated to exhibit erectile dysfunction (Wingard, et al., 2007 J. Sex Med. 4: 348-362; disc. 362-343) and broader endothelial dysfunction (Brooks-Asplund, et al., 2002 J. Appl. Physiol. 92: 2035-2044). Biochemical indicators of vascular health are characterized (arginase activity, NO concentration, and reactive oxygen species (ROS) concentration) in penile vascular tissue and aortic endothelium of diabetic ZDF and non-diabetic ZLC rats. Vascular endothelial function is measured using organ chambers in isolated rat arterial rings and in strips of erectile tissue collected from each rat strain.

### Arginase and NO Concentrations in the Type 2 Diabetic ZDF Rat Model.

ZDF rats have a mutation in the gene for the leptin receptor. When fed a high fat diet (Purina diet #5008), obese homozygous ZDF males develop hyperlipidemia and hyperglycemia by 8 weeks of age and diabetes by 12 weeks. Male ZDF rats are fed the Purina 5008 diet for 12 weeks, while ZLC controls are fed a normal diet. After 12 weeks, animals are euthanized, and aortas and penile corpora cavernosa dissected. Arginase I and II and eNOS mRNA and protein concentrations in these tissues are determined by RT-PCR and Western blot, respectively. Arginase activity is measured, as described in Example 4, by homogenizing the rat vessels in lysis buffer, removing cellular debris by centrifugation, and monitoring the hydrolysis of L-arginine using calorimetric determination of urea after the addition of isonitrosopropiophenone. The production of urea, normalized for total protein, can be used as an index for arginase activity (White, et al., 2006 Hypertension 47: 245-251) Increases in arginase activity is confined primarily to the endothelium of old rats (White, et al., 2006 Hypertension 47: 245-251) and atherogenic ApoE−/− mice (Ryoo, et al., 2008 Circ. Res. 102: 923-932). The cellular location of arginase in this model of type 2 diabetes is determined by comparing the results of RT-PCR, Western blot, and arginase activity assays in endothelial-intact and endothelial-denuded aortic rings. Findings are confirmed using immuno-histochemistry in both penile and vascular aortic rings with Arg I and II antibodies using secondary antibodies alone as a negative control. Baseline NO concentration and ROS production in ZDF vascular tissues are also measured in aortic rings and corpus cavernosum tissue, as described in Example 3, using the NO-sensitive dye DAF and the O₂ sensitive dye DHE. The same system is used to measure ROS production using the O₂ sensitive dye DHF (Ryoo, et al., 2008 Circ. Res. 102: 923-932). eNOS enzyme activity is measured directly by L-arginine to L-citrulline assay (Calbiochem-Novabiochem Corporation, La Jolla, Calif.), of aorta extracts. This assay is selective for eNOS.

The Effect of ABH on Vascular NO Production, eNOS Uncoupling and ROS Production.

To determine the effect of ABH on biochemical indicators of endothelial health in tissues explanted from ZDF or ZLC rats, aortic rings and penile corpus cavernosum strips are placed in an organ bath. ABH, at final concentrations ranging from 1 nM to 10 nM, or vehicle alone, are added to the physiological solution bathing the tissue. NO concentration are determined from cumulative DAF fluorescence (Santhanam, et al. 2007 Circ. Res. 101: 692-702). The EC50 for ABH with regard to stimulation of NO production is determined from Schild plots. To determine the extent of eNOS uncoupling, aortic rings are incubated in minimal medium overnight in the absence or presence of ABH. The nitric oxide dimer:monomer ratio is determined by Western blot. SDS-resistant eNOS dimers and monomers in aortic and penile tissue are assayed using low-temperature SDS-PAGE under nonreducing conditions. eNOS is immunoprecipitated, and the resulting samples are added to Tris glycine 6% gels (Invitrogen) without 2-mercaptoethanol. Electrophoresis is performed in an ice bath at 4° C. and the gel is stained (SimplyBlue; Invitrogen Corp.) and destained with water. ROS production, a functional indicator or eNOS uncoupling, is examined in the presence of nitro-L-arginine methyl ester (100 μM) a NOS inhibitor. If nitro-L-arginine methyl ester results in reduction in ROS production in the diabetic model, it can be concluded that eNOS is uncoupled.

### Effect of ABH on Ex Vivo Endothelial Function in ZDF Rats.

Endothelial function (vascular relaxation) is studied in aortic rings and corporal tissue in organ chambers (Brooks-Asplund, et al., 2002 J. Appl. Physiol. 92: 2035-2044), as performed in Examples 2 and 5. Tissue strips are mounted in an organ bath (Multi Myograph model 610 M, Danish Myo Technologies, Skejbyparken, Denmark) and acclimated for 30 minutes in the presence of indomethacin to control for nonspecific effects of prostacyclin-mediated inflammatory protein up-regulation. Tissues are then pre-constricted by exposure to phenylephrine (PE) at 1 μM for 10 minutes. Vascular relaxation in response to acetylcholine (ACh) is monitored after 10 minutes of precontraction with PE at 3 μM with the delivery of increasing ½ log doses of ACh every 5 minutes, starting at 1 nM and ending with 10 μM. ACh induces endothelial cell NO release in a dose-dependent fashion. Since normal initiation of erection is mediated by neuronal NOS and sustained by endothelial-dependent NO, electrical field stimulation-mediated corporal relaxation is evaluated. Electrical field stimulation (EFS) experiments are performed after 30 minutes of incubation with bretylium tosolate, a norepinephrine reuptake inhibitor (Sigma) at 30 μM and precontraction with PE at 3 μM for 10 minutes. The relaxation of the tissue to EFS delivered by a Grass S88X Stimulator (Astro-Med, West Warwich, R1, USA) is monitored during the delivery of increasing frequencies of EFS for 45 seconds at 2 milliseconds and 10 V about every 5 minutes at about 0.31, 0.62, 1.25, 2.5, 5.0, 10.0, 20.0, and 30.0 Hz. Force generation is monitored with the ADInstruments PowerLab 8/30 and interpreted by Chart 5.5.4 for Windows (ADInstruments, Colorado Springs, Colo., USA). Data is collected using a MacLab system and analyzed using Dose Response Software (AD Instruments, MA). ACh and voltage responses will both be determined in the presence and absence of ABH (1 nM to 10 nM final concentration). Schild plots are constructed to determine the EC50 of ABH with regard to both ACh (NO-dependent endothelial-mediated) and electrical field-mediated (neurogenic) vascular relaxation.

Use of the diabetic ZDF rat as a model for vascular endothelial pathology associated with type 2 diabetes will be characterized and validated. The effectiveness of ABH in improving biochemical and functional parameters of endothelial health ex vivo using this model will be evaluated. Based on preliminary data and the preliminary data of others, it is predicted that arginase activity is elevated, NO levels depressed, and ROS levels increased in the ZDF rats compared to ZLC controls and that these biochemical indicators are associated with impaired systemic vascular endothelial and erectile function. It is expected that treatment of tissue explants from diabetic ZDF rats with ABH will return these biochemical and functional parameters to values that are similar to those of ZLC rats.

### Example 7

### Effect of ABH on Vascular and Erectile Function In Vivo in a Model of Type 2 Diabetes

The effects of ABH in the ZDF rat model of type 2 diabetes after systemic in vivo administration is investigated as described below. ABH has been shown to have significant effects on both vascular and erectile function in aged rats, when given orally at approximately 200 μg (6 mg/kg) per day in drinking water for 4 weeks. Given the similarities in indicators of vascular dysfunction between aged rats and diabetic animals and humans with respect to elevated arginase and decreased NO the prediction is that orally-administered ABH will result in similar improvements in vascular and erectile function in the type 2 diabetic ZDF rats. Biochemical (arginase, NO, ROS) and functional (vasorelaxation) parameters of vascular health in vivo and ex vivo in diabetic ZDF and non-diabetic ZLC rats that have been administered ABH orally are measured, according to a protocol that previously demonstrated to be effective in aged rats. ABH is hypothesized to inhibit pathologically elevated vascular arginase, restore normal eNOS coupling, and enhance both erectile and vascular endothelial function. This study compares ZDF rats given ABH to untreated rats, and ZDF rats treated with ABH compared to ZLC rats to provide some indication of how close to healthy rats the ABH treated ZDF rats become.

ZDF rats are made diabetic by feeding a high-fat diet, while a cohort of ZLC rats are fed a normal diet. After 12 weeks, ZDF and ZLC rats are each given no ABH, 50 μg ABH per day, 100 μg ABH per day, 200 μg ABH per day, or 400 μg ABH per day for four weeks. ABH is administered orally in the drinking water. Non-invasive measurement of pulse wave velocity (PWV), as a measure of vascular health, is made at 4 week intervals throughout the entire feeding and treatment period. Aortic PWV is calculated as the separation distance divided by the difference in arrival times of the rat's pulse, with respect to the R-peak of the electrocardiogram (ECG), reported in meters per second (m/s).

At the end of the 4 week treatment period, erectile function is determined in vivo. The animals are anesthetized, and the penile crura exposed. Erectile response to electrical field stimulation (neurogenic response) is measured by catheterizing the right crus, and connecting it to a pressure transducer to permit continuous measurement of intracavernosal pressure (ICP). ICP is measured following direct stimulation of the exposed pelvic ganglion and cavernous nerve within the abdominal cavity using a square-pulse stimulator (Grass Instruments, Quincy, Mass.). A stimulation frequency of 15 Hz with a pulse width of 30 s, ranging from 2-8 V, is used. One minute stimulation periods are alternated with 2-3 minute rest periods. NO-dependent endothelial-mediated response is determined by administering ACh (an endothelial-dependent vasodilator) by injection directly into the left corpus cavernosum of the penis. The erectile response (ICP) to injections of 3, 10, and 30 mg is determined. ICP is monitored during ACh administration, and until it returns to baseline. A waiting period of 10-15 minutes, from the end of the previous response, is used between injections. For both neurogenic and endothelium-dependent stimulation, total erectile response or total ICP is determined by the area under the erectile curve (AUC; mmHg/sec) from the beginning of CNS until the ICP pressure returns to baseline or pre-stimulation pressures. During stimulation, the peak ICP (PICP) is registered from the level of the BICP. The ratio between the maximal ICP and MAP obtained at the peak of erectile response is calculated to normalize for variations in systemic blood pressure. Rats are euthanized, aortic and penile corpus cavernosum tissue dissected, and endothelial dependent vasorelaxant responses are determined using electrical field stimulation in an organ bath, as described in Example 6. Aortic and penile vascular tissues from rats is also analyzed for arginase and eNOS levels, arginase activity and for NO and ROS production, as described in Example 6.

This will evaluate the effect of systemically-administered ABH on parameters of vascular endothelial function in type 2 diabetic ZDF rats and validate the effects of ex vivo treatment with ABH. We expect that diabetic ZDF rats treated with ABH will demonstrate significant improvements in in vivo erectile function in response to cavernous nerve stimulation and intra-cavernous ACh administration, pulse wave velocity and in ex vivo vasorelaxation following electrical field stimulation of cavernosal tissue and Ach treatment of aortic rings, compared with untreated ZDF rats. Furthermore, these improvements are anticipated to be associated with significant decreases in vascular arginase activity and vascular ROS concentration, and with elevations in eNOS activity indicated by increased NO concentration compared with untreated diabetic ZDF rats. We expect that ABH treatment should result in values for each of these functional and biochemical endothelial parameters that are similar to those observed in non-diabetic ZLC rats. These expectations are based on previous observations in aged rats, which, after a similar treatment protocol, exhibited vascular function and erectile responses that were comparable to those of young rats. While there can be a small improvement in vascular and erectile function in the ZLC control rats, the effect and improvement in the ZDF rats is considerably larger and more significant. Erectile dysfunction is increasingly being regarded as an early indicator of more generalized cardiovascular pathology. As a secondary, although not insignificant outcome small improvements in systemic vascular stiffness (as determined by PWV) can be observed in ABH-treated diabetic ZDF rats compared with untreated ZDF rats. Similar improvements have been demonstrated in vascular stiffness in aged rats.

### Example 8

### Synergy Between ABH and PDE5 Inhibitors on Vascular and Erectile Function

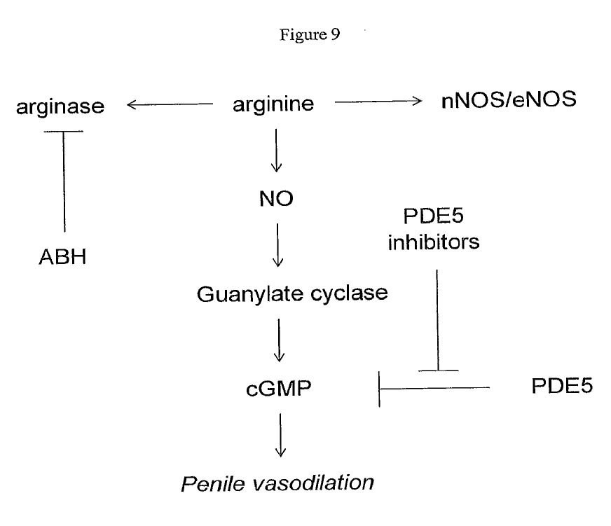

Phosphodiesterase type 5 (PDE5) inhibitors have proven to be an incredibly successful treatment for mild erectile dysfunction (ED) in a large number of patients. PDE5 inhibitors work by slowing the degradation of cyclic GMP (cGMP), a downstream signaling effector of NO, in erectile and other vascular beds. Patients with severe ED, including men with diabetes, respond poorly to PDE5 inhibitors (Rendell, et al., 1999 JAMA 281: 421-426). The most likely reason for this is that when NO is depleted because of increased arginase activity (as we propose is the case in diabetic vasculopathy), drugs that modify downstream signaling of NO are ineffective. At the present time, pharmacological agents which improve NO-biosynthesis in the penis and thus increase corporal cGMP concentrations and potentially improve the effectiveness of PDE5 inhibitors are lacking. If ABH decreases vascular arginase activity, and increases NO concentration, that it might also increase responsiveness to PDE5 inhibitor therapy, and that ABH and PDE5 inhibition might therefore exert a synergistic effect on improving vascular health and erectile function. FIG. 9 is a schematic representation of synergistic interaction between ABH and PDE5 inhibitors. The effects of ABH and PDE5 inhibitors, singly and in combination, in the ZDF model of type 2 diabetes are tested. ZDF rats have previously been found to be relatively non-responsive to PDE5 inhibitors, compared to ZLC rats (Russo, et al., 2008 Endocrinology 149: 1480-1489), suggesting their appropriateness in the proposed study.

ZDF rats are made diabetic according to the feeding protocol described in Example 6. ZLC rats are maintained on a normal diet. Rats will then be administered either ABH at the lowest effective dose determined in Example 7, or vehicle alone, for four weeks in their drinking water. Following this, animals are subjected to acute in vivo erectile function testing, as described in Example 7. Ten to twenty minutes prior to each in vivo vasorelaxation experiment, animals will receive a single intravenous dose of the PDE5 inhibitor, sildenafil citrate (Viagra; either 1 or 2 mg/kg iv) or vehicle alone. These doses were chosen from preliminary experiments and our previously published results (Bivalacqua, et al., 2004 Int. J. Impot. Res. 16: 21-29). Peak intracavernous pressure, ICP/mean arterial pressure (MAP), and total ICP (area under the erectile curve) are determined in response to intra-cavernous administration of the endothelium-dependent vasodilator ACh, and in response to direct cavernous nerve stimulation. Animals will then be euthanized, and aortas and penile vascular tissues are collected. Biochemical (arginase, NO, eNOS, ROS) and functional (contraction and relaxation in organ baths) indicators of endothelial health are determined in aortic rings and cavernous strips.

It is expected that diabetic ZDF rats receiving both ABH and the PDE5 inhibitor will exhibit significant improvements in all parameters of erectile function in vivo and vasorelaxation ex vivo compared to rats receiving only one drug, or no drug. PDE5 inhibition enhances nitric oxide-induced vasorelaxation by increasing smooth muscle cGMP concentration. In the ZDF rat, we believe that ABH will improve endothelial-derived NO biosynthesis in the penile vasculature, thus significantly improving peak ICP erectile responses, while the combination of ABH and PDE5 inhibition can improve all penile hemodynamic measures studied (i.e. peak ICP and total ICP) further. Previous studies suggest that diabetic ZDF rats are unlikely to respond to PDE5 inhibitors alone, although, as an “upstream” therapeutic, the expectation is that ABH alone will exert some positive effects. Any differences in erectile function observed in ZLC rats treated with ABH alone or in combination with the PDE5 inhibitor are anticipated to be much smaller than those observed in diabetic ZDF rats.

### Example 9

### Synergy Between ABH and PDE5 Inhibitors on cGMP Levels in Tissues Tissue Sample Collection

The source of tissue was old (22 to 24 months) male Fischer 344 rats purchased from National Institute for Aging (NIA). The rats were anesthetized in an isoflurane chamber and euthanized. The entire length of the aorta (thoracic, arch, abdominal) were dissected out and cleaned free of connective tissue. These aorta were then sectioned into ˜3 mm long rings. Samples were collected from 2 rats and pooled. A total of 25 rings were prepared.

### Cell Treatment

Rings were incubated in varying concentrations of sildenafil (0-2000 nM) and ABH (0-500 nM) prepared in 200 μL Krebs buffer for 10 min at 37° C. The rings were then snap frozen in liquid nitrogen following treatment. The next day, samples were homogenized in 200 μL 6% TCA buffer (prepared by adding TCA to DI water) at 4° C. for extraction of cyclic nucleotides. The samples were then centrifuged at 12,000 rpm for 5 min at 4° C. Protein pellets were re-dissolved in PBS to determine protein concentration, and supernatant was reserved for cGMP detection. The supernatant was extracted with ether to remove the TCA, dried and then re-suspended in assay buffer as provided by manufacturer for assay performance.

### Solutions of ABH and Sildenafil are Described Below:

ABH (MW 211.45): A stock solution of ABH at a concentration of 100 mM was prepared by dissolving 21.1 mg of ABH into 1 ml Krebs solution. This was used to prepare 5 ml of a 1 mM stock. 200 μL of the 1 mM solution were dispensed into column 12 of a 96-well plate. Six 2× dilutions were prepared in the plate. 200 μL Krebs were dispensed in column 5 to get a concentration range of 0-1000 nM (2× of final concentration).

Sildenafil (MW 474.5): A stock solution of sildenafil at a concentartion of 100 mM was prepared by dissolving 47.4 mg of sildenafil into 1 ml Krebs solution; 5 ml of 2 mM stock was prepared from this. Row A received 200 μL of Krebs buffer (0 mM sildenafil), mixed well, and adjusted to a final volume of 200 μL. Next, 200 μL of the 2 mM sildenafil stock was dispensed into row H of the 96 well plate already containing the ABH dilutions (0-4000 nM). Six 2× dilutions were prepared from this to get the final concentration ranges for the two drugs.

cGMP Assay

cGMP levels were determined with a cGMP kit (Amersham cGMP Enzyme immunoassay Biotrak (EIA) System Cat #RPN226) and run in accordance with the manufacture's recommendations. The assay uses a competitive fluorescent immunoassay format to measure levels of cGMP. It combines the use of a peroxidase-labelled cGMP conjugate, a specific antiserum which can be immobilized on to pre-coated microplates, and a one-pot stabilized substrate solution. Tissue extracts were transferred to eppendorf tubes, centrifuged and kept on ice. 40 μl of the lysate was added to a 96 well microplate coated with Goat anti Rabbit IgG, followed by addition of HRP labeled cGMP conjugate and rabbit anti-cGMP antibody . In the absence of cGMP most of the HRP conjugate is bound to the antibody and yields a high fluorescence. Increasing concentrations of cGMP competitively decreases the binding of the HRP conjugate, decreasing the measured fluorescence (HRP activity). A standard curve for the concentration of cGMP ranging from 400 to 0.016 pmol was run with every assay (400, 4, 1.3, 0.44, 0.15, 0.049, 0.016,0 pmol). The plate was then incubated for 2 hrs at room temperature with shaking after which it is washed and the fluorescent substrate is added. The plate was incubated for an additional 10 min and then read on a Molecular Devices Gemini EM plate reader set at Ex 530 nm, Em 590 nm (endpoint mode). The signal is reported to be stable for up to 24 hrs, but was generally read within 2 hrs.

### Experimental Results and Conclusions:

Table 2 shows the number of pmols of cGMP/mg protein produced from the cGMP assay, which was conducted in triplicate. These values were calculated based on the resultant ratio of fluorescence reading of sample divided by fluorescense reading with no cGMP (pmols/mg). For each concentration combination of sildenafil and ABH administered to cells, absorbance (OD at 450 nm) was measured. From these values, the absorbance adjusted for non-specific binding (NSB) for each sample (OD-NSB(B)) was determined from the absorbance (B) divided by the absorbance without cGMP (Bo). Using this value and the standard curve for cGMP, the log (fmol) is found and, after adjusting for the quantity of protein, the pmols of cGMP/mg protein is calculated.

TABLE 3
Numer of pmols of cGMP/mg protein produced
from combinations of sildenafil with ABH

| | | sildenafil (nM) | | | | |
|---|---|---|---|---|---|---|
| | P(x, y) | 0 | 31 | 125 | 500 | 2000 |
| ABH | 0 | 0.04 | 0.27 | 2.56 | 0.88 | 0.80 |
| (nM) | 7.81 | 0.34 | 1.99 | 1.45 | 0.13 | 2.18 |
| | 31.25 | 0.90 | 2.84 | 2.13 | 1.79 | 4.79 |
| | 125 | 1.14 | 1.27 | 1.11 | 5.25 | 4.91 |
| | 500 | 0.16 | 2.31 | 1.61 | 2.10 | 4.32 |

The mean resulting pmols/mg (P) for the three data points for each concentration combination are arrayed in Table 3. Notably, row 1 represents treatment with sildenafil alone at various concentrations without ABH and column 1 represents treatment with ABH alone at various concentrations without sildenafil. P(x,y) represents mean pmols/mg resulting from treatment with concentration of x units of sildenafil and y units of ABH.

TABLE 4
Theoretical response, pmol/mg, based on an additive effect

| | | sildenafil (nM) | | | | |
|---|---|---|---|---|---|---|
| | T(x, y) | 0 | 31 | 125 | 500 | 2000 |
| ABH | 0 | 0.04 | 0.27 | 2.56 | 0.88 | 0.80 |
| (nM) | 7.81 | 0.34 | 0.57 | 2.86 | 1.18 | 1.10 |
| | 31.25 | 0.90 | 1.12 | 3.42 | 1.74 | 1.65 |
| | 125 | 1.14 | 1.37 | 3.67 | 1.98 | 1.90 |
| | 500 | 0.16 | 0.39 | 2.68 | 1.00 | 0.92 |

Table 4 shows the theoretical values (T(x,y)) for the amount of pmols/mg produced if the relationship between ABH and sildenafil were additive. It is calculated as T(x,y)=P(x,0)+P(0,y)−P(0,0).

TABLE 5
Measurement of the amount of synergy

| | | sildenafil (nM) | | | | |
|---|---|---|---|---|---|---|
| | D(x, y) | 0 | 31 | 125 | 500 | 2000 |
| ABH | 0 | | | | | |
| (nM) | 7.81 | | 1.42 | −1.42 | −1.05 | 1.09 |
| | 31.25 | | 1.72 | −1.29 | 0.06 | 3.14 |
| | 125 | | −0.10 | −2.55 | 3.26 | 3.01 |
| | 500 | | 1.93 | −1.07 | 1.10 | 3.40 |

Table 5 measures the quantum of synergy by comparing the difference D(x,y) between observed P(x,y) with the theoretic T(x,y). It is calculated as D(x,y)=P(x,y)−T(x,y). Positive numbers reflect the increase in pmols/mg from the combination of sildenafil and ABH for any given x,y.

TABLE 6
Percentage change in pmols/mg observed

| | | sildenafil (nM) | | | | |
|---|---|---|---|---|---|---|
| | PC (x, y) | 0 | 31 | 125 | 500 | 2000 |
| ABH | 0 | | | | | |
| (nM) | 7.81 | | 250% | −49% | −89% | 99% |
| | 31.25 | | 153% | −38% | 3% | 190% |
| | 125 | | −8% | −70% | 165% | 158% |
| | 500 | | 499% | −40% | 110% | 370% |

Table 6 measures the percentage change in pmols/mg observed PC (x,y). It is calculated ac PC(x,y)=D(x,y)/T(x,y).

The data in Tables 5 and 6 demonstrate that synergy was observed in many of the concentration combinations analyzed, yielding results as much as 499% greater than would be expected if the combined effect of ABH and sildenafil was additive. The encouraging results from this preliminary study in rat aortic tissues prompted additional research into synergistic combinations of ABH and sildenafil, such as the use of human cell lines as outlined below.

### Example 10

### Synergy Between an Arginase Inhibitor and Sildenafil Inhibitors on cGMP Levels in Cells

The following procedure can be used to show if there is synergy when cells are treated with an arginase inhibitor and a phosphodiesterase inhibitor, such as sildenafil.

### Cell Preparation

An aliquot of pulmonary artery endothelial (PAEC) cells from a single donor of a species such as human, porcine, bovine, and ovine can be obtained. These cells may be obtained commercial sources. For example human pulmonary artery endothelial (HPAEC) cells are obtained frozen from Lonza (Cat# CC-2530). Samples obtained frozen are, thawed, sub-cultured and maintained following the manufacturer's instructions. Cells are subcultured when they are 70-80% confluent and contain many mitiotic figures throughout the flask. Cells are fed every other day by removing the existing media and replacing with new media.

### Cell Treatment

PAEC cells are seeded in 24 well plates at a density of 3×10⁵ cells/well in Lonza's cell specific growth media (Cat # CC-3162) containing 7.5 mg/dl of uric acid (Sigma) and incubated overnight at 37° C. in 5% CO₂. The media is aspirated and 900 or 800 μl of HBSS is added to each well depending on whether one or two compounds respectively are being added to the wells. Dose curve concentrations of the compound of interest are made in HBSS buffer and the appropriate volume is added to each well to achieve the necessary final concentration as shown below.

Arginase Inhibitor: A stock solution of 1 M ABH is prepared by adding 52 mg of the compound to 0.246 mls of Hank's Balanced Salt Solution (HBSS). 100 μl of 10× concentrations from a dilution series of 1×10⁻⁴ to 1×10⁻⁷ M, made from the stock solution, is added to HBSS in each experimental well. This addition results in final concentrations of 1×10⁻⁶ to 1.25×10⁻⁸ M of ABH.

Sildenafil: 100 mg is added to 2.10 ml of 100% DMSO to give a 100 mM stock which is further diluted 1000 fold in HBSS to give working stock of 1×10⁻⁴ M. 100 μl of 10X concentrations from a dilution series of 5×10⁻⁴ to 1×10⁻⁷ M, made from the stock solution, is added to the HBSS in each experimental well. This results in final concentrations of 2×10⁻⁶ to 1×10⁻⁸M Sildenafil. If both drugs are to be added for combination studies then 800 ul of HBSS is added to each well

The HBSS is then completely aspirated and the cells are lysed in 200 μl lysis buffer, provided by the cGMP kit, for 15 min at room temperature. Lysis is monitored visually under a microscope and a protein determination is performed to confirm that a uniform lysis occurred.

cGMP Assay

cGMP levels are determined with a cGMP kit (such as Molecular Devices Cat #R8074) and run in accordance with the manufacture's recommendations. The assay use a competitive fluorescent immunoassay format to measure levels of cGMP. Cell lysates are transferred to eppendorf tubes, centrifuged and kept on ice. 40 μl of the lysate is added to a 96 well microplate coated with Goat anti Rabbit IgG, followed by addition of HRP labeled cGMP conjugate and rabbit anti-cGMP antibody. In the absence of cGMP most of the HRP conjugate is bound to the antibody and yields a high fluorescence. Increasing concentrations of cGMP competitively decrease the binding of the HRP conjugate, decreasing the measured fluorescence (HRP activity). A standard curve for the concentration of cGMP is run with every assay with concentrations ranging from 400 to 0.016 pmol For example, 400, 4, 1.3, 0.44, 0.15, 0.049, 0.016, and 0 pmol are used. The plate is then incubated for 2 hrs at room temperature with shaking after which it is washed and the fluorescent substrate is added. The plate is incubated for an additional 10 min and then read on a plate reader, such as a Perkin Elmer Victor 1 plate reader set at Ex 530 nm, Em 590 nm (endpoint mode). The signal is reported to be stable for up to 24 hrs, but is generally read within 2 hrs.

Although the preceding example used uric acid-treated hPAECs as a mechanism to induce arginase and thereby act as an in vitro surrogate model for Pulmonary Aterial Hypertension (PAH), there are a number of other endothelial cell (EC) lines that could be used that would serve as potential cellular models and surrogates for other diseases under appropriate conditions. These cell lines and the associated diseases that they might be used to study are listed in Table 6. Obviously, one needs to be assured that the cell system has the requisite molecular targets that result in cGMP concentrations. Thus, the cell system needs to possess upregulated arginase (to model the diseased state), a phosphodiesterase of the type 1, 2 and/or 5 (PDE1, PDE2, and/or PDE5), guanylate cyclase and eNOS. Not all ECs contain all of these components and thus, in some cases, the endothelial cells may need to be co-cultured with smooth muscle cells (SMC) which contain guanylate cyclase and PDE5 in order to assess cGMP levels. Furthermore, uric acid is not the only agent that can be used to upregulate arginase. It has also been shown that thrombin treatment increases arginase activity in PAECs [Ming et al. 2004, Circ. 110:3708-3714]. In addition, arginase can be activated in endothelial cells by atherogenic lipids such as OxLDL, simulating models of atherosclerosis [Ryoo et al., 2006, Circ Res 99:951-960].

TABLE 6
Table of Endothelial Cell Lines.

| Cell Type | Abbreviation | Disease |
|---|---|---|
| Human Aortic Endothelial | | 1) Atherosclerosis |
| HAECs Cells | | 2) Peripheral Vascular Disease |
| Human Coronary | HCECs | 1) Coronary Artery Disease |
| Endothelial Cells | | |
| Human Pulmonary Arterial | HPAECs | 1) Pulmonary Hypertension |
| Endothelial Cells | | |
| Human Umbilical Vein | HUVECs | 1) Thromboembolic Disease |
| Endothelial Cells | | |
| Human Microvascular | HMECs | 1) Systemic Hypertension |
| Endothelial Cells | | 2) Diabetic vasculopathy |
| Human Dermal | HDMECs | 1) Raynaud's disease |
| Microvascular Endothelial | | 2) Vasculopathy of connective |
| Cells | | tissue diseases |
| | | 3) Wound healing and Diabetic |
| | | microvascular disease |
| Human Uterine | UtMVEC | 1) Pregnancy Induced |
| Microvascular ECs | | Hypertension (Pre-eclampsia) |
| Human Iliac Artery ECs | HIAEC | 1) Peripheral Vascular Disease |
| Human Cardiac | HMVEC | 1) Heart Failure |
| Microvascular ECs | | |

Once the synergistic combination of an arginase inhibitor and a phosphodiesterase inhibitor has been optimized in cellular, tissue, or organ bath assays, the use of such a combination can be further validated in animal models of these diseases associated with endothelial dysfunction.

Thus, a synergistic combination of an arginase inhibitor and a phosphodiesterase inhibitor for the treatment of asthma could be validated in the mouse, guinea pig, or monkey model of allergic asthma. The animals can be sensitized to various allergens including but not limited to ovalbumin or Dermatophagoides farinae (dust mites). Arginase inhibitors have already been shown to have significant effects as single agents in allergen-induced animal models of asthma [Maarsinghe et al., 2009, Br J Pharmacol 158:652-664].

A synergistic combination of an arginase inhibitor and a phosphodiesterase inhibitor for the treatment of erectile dysfunction (ED) can be evaluated by measuring penile intracavernous pressure following electrical stimulation of the pelvic nerve. ED is associated with aging, diabetes, and atherosclerosis. For atherosclerosis-associated ED, hypercholesterolemic rabbits or ApoE KO mice on a Western diet can be utilized [Behr-Roussel et al., 2006, J Sex Med 3:596-603]. For diabetes-associated ED, alloxan- or streptozotocin-treated animals can be used for type-1 diabetes or Zucker Diabetic Fatty rats for type 2 diabetes (as described in Example 8). As single agents, PDE5 inhibitors have been approved for treatment of ED, although there is a significant population of non-responders associated with diabetes. Arginase inhibitors as well as arginase I antisense have been shown to be effective in restoring erectile response in aged rat penis [Bivalacqua et al., 2007, Am Physiol Heart Circ Physiol 292:H1340-H1351].

In a similar manner, female sexual dysfunction may be treated with a synergistic combination of an arginase inhibitor and a phosphodiesterase inhibitor. As single agents, each of these agents has been shown to increase vaginal blood flow in a rabbit model following pelvic nerve stimulation [Kim et al., 2003, Intl J Impotence Res 15:355-361].

A synergistic combination of an arginase inhibitor and a phosphodiesterase inhibitor for the treatment of pulmonary arterial hypertension (PAH) can be evaluated in several different experimental animal models. These models include the use of pathophysiological stimuli (hypoxia, increased flow, and vascular obstruction), chemical-induction (monocrotaline (MCT), α-naphthylthiourea, bleomycin, and Group B streptococcus), molecular-stimuli (VEGF receptor inhibition plus hypoxia or Angiopoietin-1 overexpression), and genetic stimuli (sickle-cell (SS) mice, fawn-hooded rat, broiler chicken, BMPR2 KO mice, and S100A4 overexpression in mice). As single agents, several PDE5 inhibitors have already been approved for treatment of PAH.

Emerging evidence suggests that arginase contributes to the oxidative stress and vascular endothelial dysfunction seen in women with pre-eclampsia [Sankaralingam et al., 2009, Cardiovascular Research 54:897-904]. There is also evidence that PDE5 inhibitors may attenuate the endothelial dysfunction seen with this pregnancy induced hypertension [Turgut et al., 2008, Eur J Pharmacol 589:180-187]. The synergistic interaction of arginase and PDE5 inhibition can be evaluated in a cellular endothelial model using human uterine microvascular endothelial cells. In addition this synergy can be studied in a newly established rat model of pre-eclampsia, the suramin treated rat model [Nash et al., 2005, Placenta 26:410-418].

Arginase is constitutively expressed in endothelial cells which form the lining of the entire circulatory system from the heart to the smallest capillary. Therefore, the number of cardiovascular disorders which could be treated with arginase inhibitors is diverse including hypertension, peripheral vascular disease (PVD), peripheral arterial disease (PAD) or intermittent claudication, coronary artery disease (CAD), Raynaud's disease, and cardiovascular complications such as myocardial infarction or stroke. However, most of the cardiovascular disorders can be attributed to the presence of atherosclerosis, the build-up of vulnerable plaques in the vascular circulation. A synergistic combination of an arginase inhibitor and a phosphodiesterase inhibitor for the treatment of atherosclerosis can be evaluated in animal models such as the ApoE knock-out mice fed a high-cholesterol diet or hpercholesterolemic rabbits. Arginase inhibitors have been effective in the ApoE KO model [Ryoo et al., 2008, Circ Res 102:923-932].

### APPENDIX A

### Structures of Various Inhibitors of the Invention

### PDE1 Inhibitors

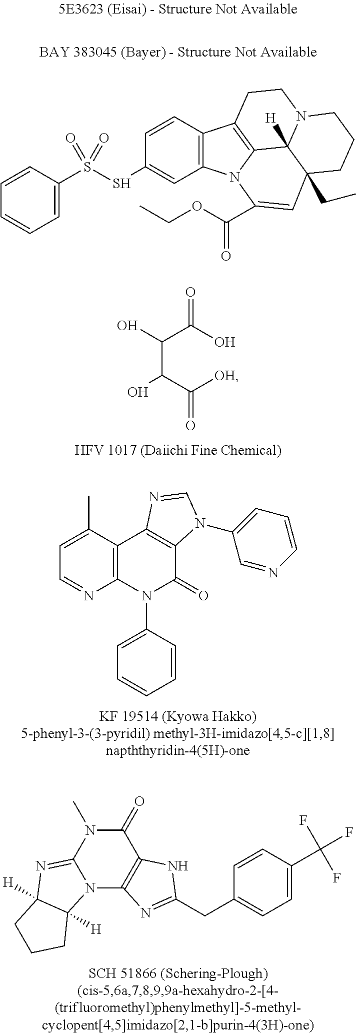

### PDE2 Inhibitors

BAY607550 (Bayer).-2-(3,4-dimethoxybenzyl)-7-{(1R)-1-[(1R)-1-hydroxyethyl]-4-phenylbutyl}-5-methylimidazo[5,1-f][1,2,4]triazin-4(3H)-one

### PDE5 inhibitors

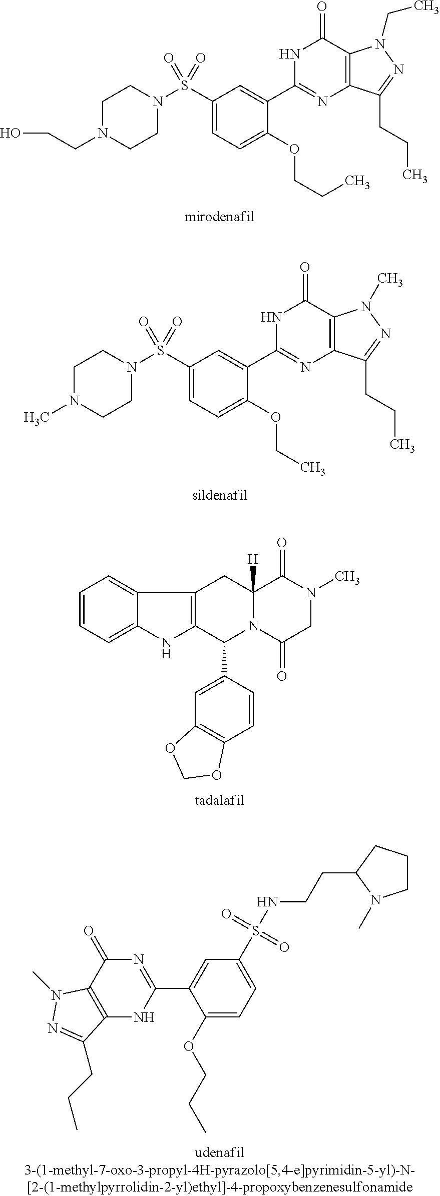

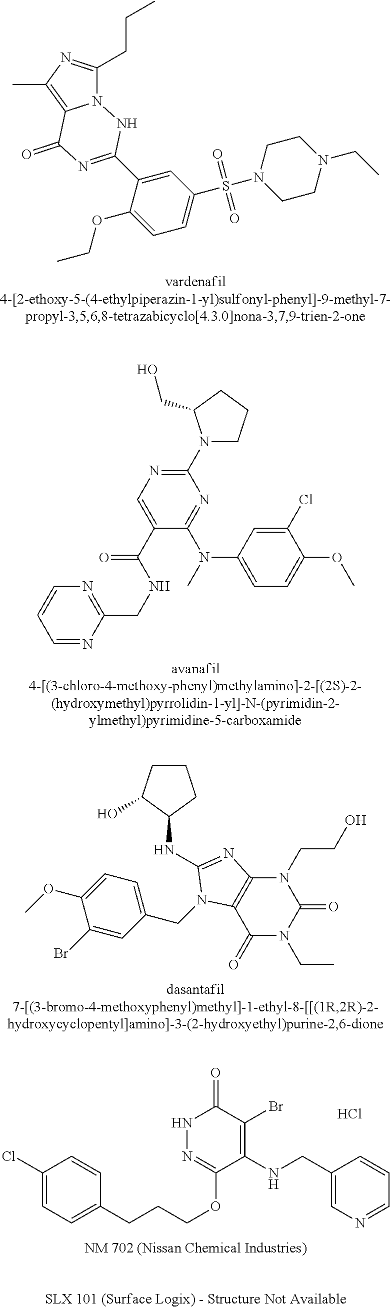

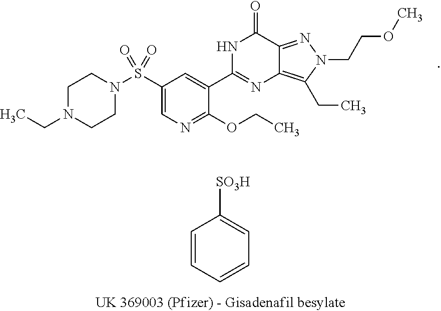

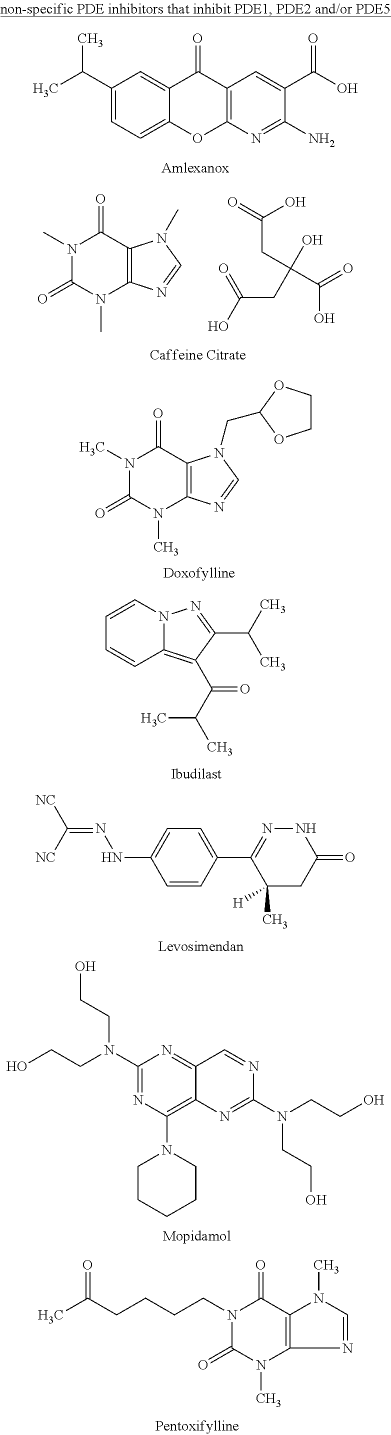

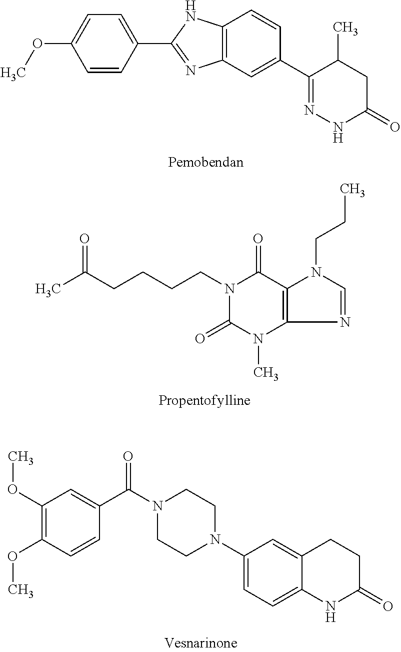

1. A composition comprising a therapeutically effective amount of a synergistically-effective combination of at least one arginase inhibitor and at least one phosphodiesterase (PDE) inhibitor formulated in a physiologically-acceptable pharmaceutical medium.

2. The composition of claim 1, wherein the at least one arginase inhibitor is 2(S)-Amino-6-boronohexanoic acid (ABH), S-(2-boronoethyl)-L-cysteine (BEC), N^{ω}-hydroxy-nor-L-arginine (nor-NOHA), N^{ω}-hydroxy-L-arginine (NOHA), or a combination thereof.

3. The composition of claim 1, wherein the at least one PDE inhibitor is a PDE1 inhibitor, a PDE2 inhibitor, a PDE5 inhibitor, a non-specific PDE inhibitor that inhibits PDE1, PDE2 and/or PDE5, or a combination thereof.

4. The composition of claim 3, wherein the PDE 1 inhibitor is 5E3623, BAY 383045, HFV 1017, KF 19514, SCH 51866, or a combination thereof.

5. The composition of claim 3, wherein the PDE2 inhibitor is BAY 607550.

6. The composition of claim 3, wherein the PDE5 inhibitor is mirodenafil, sildenafil, tadalafil, udenafil, vardenafil, avanafil, dasantafil, NM 702, SLX 101, UK 369003, or a combination thereof.

7. The composition of claim 3, wherein the non-specific PDE inhibitor that inhibits PDE1, PDE2 and/or PDE5 is amlexanox, caffeine citrate, doxofylline, levosimendan, mopidamol, pentoxifylline, pemobendan, propentofylline, vesnarinone, ibudilast , or a combination thereof.

8. A method of treating an endothelial disorder, the method comprising administering to a patient in need thereof a synergistically-effective amount of at least one arginase inhibitor and at least one phosphodiesterase (PDE) inhibitor.

9. The method of claim 8, wherein the endothelial disorder is asthma, a cardiovascular disorder, erectile dysfunction, female sexual dysfunction, inflammation, intermittent claudication, a peripheral arterial occlusive disorder, pulmonary hypertension, Raynaud's disease, stroke or systemic hypertension.

10. The method of claim 8, wherein the at least one arginase inhibitor is 2(S)-Amino-6-boronohexanoic acid (ABH), S-(2-boronoethyl)-L-cysteine (BEC), N^{ω}-hydroxy-nor-L-arginine (nor-NOHA), N^{ω}-hydroxy-L-arginine (NOHA), or a combination thereof.

11. The method of claim 8, wherein the at least one PDE inhibitor is a PDE1 inhibitor, a PDE2 inhibitor, a PDE5 inhibitor, a non-specific PDE inhibitor that inhibit PDE1, PDE2 and/or PDE5, or a combination thereof.

12. The method of claim 12, wherein the PDE 1 inhibitor is 5E3623, BAY 383045, HFV 1017, KF 19514, SCH 51866, or a combination thereof.

13. The method of claim 12, wherein the PDE2 inhibitor is BAY 607550.

14. The method of claim 12, wherein the PDE5 inhibitors is mirodenafil, sildenafil, tadalafil, udenafil, vardenafil, avanafil, dasantafil, NM 702, SLX 101, UK 369003, or a combination thereof.

15. The method of claim 12, wherein the non-specific PDE inhibitor that inhibits PDE1, PDE2 and/or PDE5 is amlexanox, caffeine citrate, doxofylline, levosimendan, mopidamol, pentoxifylline, pemobendan, propentofylline, vesnarinone, ibudilast, or a combination thereof.

16. The method of claim 8, wherein the synergistically-effective amount of at least one arginase inhibitor and at least one phosphodiesterase (PDE) inhibitor is administered together in a single composition.

17. The method of claim 8, wherein the synergistically-effective amount of at least one arginase inhibitor and at least one phosphodiesterase (PDE) inhibitor is administered in separate compositions.

18. The method of claim 8, wherein the endothelial disorder is asthma and the at least one arginase inhibitor and at least one phosphodiesterase (PDE) inhibitor are administered by at least one of oral, inhalation, intranasal and topical.

19. The method of claim 8, wherein the endothelial disorder is erectile dysfunction or female sexual dysfunction and the inhibitors are administered via orally, topically or by injection.

20. The method of claim 8, wherein the endothelial disorder is a cardiovascular disorder and the inhibitors are administered orally.

21. A regime or regime for treating an endothelial disorder, the regime or regime comprising administering to a patient in need thereof a synergistically-effective amount of at least one arginase inhibitor and at least one phosphodiesterase (PDE) inhibitor for a specified time at a specified dosing schedule.

22. A kit comprising a formulation comprising a unit dose of at least one arginase inhibitor, and at least one PDE inhibitor, or combination thereof, and a pharmaceutically-acceptable excipient to administer the dosage form according to a desired regimen or exemplary regimen , said kit optionally comprising instructions for the use of the kit.

23. (canceled)

24. (canceled)
